SEQUENCE LISTING (11 sequences)

SEQ ID NO: 1 - DNA, 24 bp, Artificial Sequence
  A synthetic primer
ctggcttgtttagatacagtagtc

SEQ ID NO: 2 - DNA, 22 bp, Artificial Sequence
  A synthetic primer
cttgtgataggcctagttgatt

SEQ ID NO: 3 - DNA, 21 bp, Artificial Sequence
  A synthetic primer
atatcagctgtaaagcctggg

SEQ ID NO: 4 - DNA, 25 bp, Artificial Sequence
  A synthetic primer
gttgtcaatatcctggaaattttgc

SEQ ID NO: 5 - DNA, 26 bp, Artificial Sequence
  A synthetic primer
ctgccattctttagaggggatgcttg

SEQ ID NO: 6 - DNA, 31 bp, Artificial Sequence
  A synthetic primer
ggggacaagtttgtacaaaaaagcaggctnn

SEQ ID NO: 7 - DNA, 30 bp, Artificial Sequence
  A synthetic primer
ggggaccactttgtacaagaaagctgggtn

SEQ ID NO: 8 - DNA, 30 bp, Artificial Sequence
  A synthetic primer
nnnacaagtttgtacaaaaaagcaggctnn

SEQ ID NO: 9 - DNA, 30 bp, Artificial Sequence
  A synthetic primer
nnagcctgcttttttgtacaaacttgtnnn

SEQ ID NO: 10 - DNA, 33 bp, Artificial Sequence
  A synthetic primer
nnnnacccagctttcttgtacaaagtggtnnnn

SEQ ID NO: 11 - DNA, 33 bp, Artificial Sequence
  A synthetic primer
nnnnaccactttgtacaagaaagctgggtnnnn

(19) United States
(12) Patent Application Publication
(10) Pub. No.: US 2004/0132042 A1
(43) Pub. Date: Jul. 8, 2004

(54) Use of double and opposite recombination sites or the single step cloning of two dna segments

(76) Inventors: Valerie Marie-Noelle Frankard, Brussel (BE); Gonda Paola Julie Coosemans, Gent (BE); Chris De Wilde, Herdersem (BE)

Correspondence Address: Schwegman Lundberg, Woessner & Kluth, PO Box 2938, Minneapolis, MN, 55402, US

(21) Appl. No.: 10/474,288

(22) PCT Filed: Apr. 3, 2002
(86) PCT No.: PCT/EP02/03652

(30) Foreign Application Priority Data
Apr. 6, 2001 (EP) 01870077.3

(51) Int. Cl. C12Q 1/68; C12N 15/85

(52) U.S. Cl. 435/6; 435/69.1; 435/455; 435/320.1; 435/325

(57) ABSTRACT

The present invention relates to the easy cloning of multiple DNA fragments at multiple places in the vector by a single step recombination reaction. More specifically the present invention discloses the use of two recombination sites each having the same pair of recombination sequences and the recombination sites are placed in opposite direction, in order to prevent the recombination between the sites themselves, and offering the opportunity to clone different or the same DNA fragments in multiple sites of the vector. This method is very useful for high throughput cloning of for example a co-suppression vector, or a gene combination vector, or a promoter combination vector or a promoter-gene combination vector, or a gene silencing vector, or a polycistronic RNA vector, or a gene stacking vector, or a biderectional promoter, or combinatorial expression cassettes or a fusion protein.

### FIELD OF INVENTION

The present invention relates to molecular biology, more particularly to the design and use of novel cloning vectors for the single-step, simultaneous and directional insertion of two DNA fragments in said vectors. Herein disclosed is the use of site-specific recombination sites which have the same pair of recombination sequences, said recombination sites placed in opposite directions in the cloning vectors. The cloning vectors of the present invention find application in the high-throughput construction of two-insert expression vectors such as for example co-suppression vectors, gene silencing vectors, polycistronic vectors, gene combination vectors, promoter combination vectors or promoter-gene combination vectors. Said vectors or parts thereof, can ultimately be utilized for driving the desired effect of a cloned DNA inserts in a host organism, like a transgenic plant.

### BACKGROUND

Molecular biology tools are available for cloning DNA fragments from one organism into vectors for introducing those DNA fragments in other organisms. This so-called recombinant DNA technology became very important in modem research areas in both the medical and agricultural world. As the range of applications of this technology is extending every day, the need for more efficient cloning techniques is growing accordingly.

In classical cloning, a typical cloning round comprises the time consuming steps of isolating, analysing, purifying, manipulating and assembling the DNA and involves the use of restriction endonucleases, ligase enzymes, and optionally also other DNA modifying enzymes such as T₄-DNA polmerase, Klenow fragment, kinases or dephosphorylases (Sambrook et al. (1989), Molecular cloning: A laboratory manual. Cold Spring Harbor Laboratory press).

Furthermore, the use of restriction enzymes that cut the DNA at particular sites is sometimes cumbersome. For example, the choice of usable restriction enzymes for cloning DNA sequences is limited when the size of the DNA fragment Increases, since the number of internal restriction sites increases. The use of restriction enzymes is also inconvenient when cloning DNA fragments of unknown sequence, also because of the risk of cutting the DNA in an internal restriction site. Thus rare (and mostly very expensive) restriction enzymes may be necessary to clone large segments of DNA or fragments with unknown sequence. Moreover, prior to cloning the DNA segment, it is often necessary to introduce suitable restriction site in the DNA segment via the process of mutagenesis. Finally, the classical cloning techniques do not allow very complex cloning strategies, meaning that seldom more than 4 fragments can be ligated in one reaction. For more complex cloning, intermediate constructs have to be made in multiple rounds of cloning.

Consequently, novel cloning techniques not based on the use of restriction enzymes are less limiting and less labor intensive then the classical methods. Most importantly, cloning techniques that allow a single step cloning of multiple DNA fragments, constitute a large advantage over the classical used techniques.

As often complex vectors or parts thereof have to be constructed for modern gene technology applications, techniques have been developed to handle more complex, large and unknown DNA fragments.

Recently, novel cloning strategies have been developed that are based on the use of site-specific recombination enzymes. These techniques do not rely on the use of restriction enzymes and ligases and can be used for easy cloning of large DNA fragments.

The site-specific cloning method of Gateway™ (www.lifetech.com/gateway) is based on the use of specially designed recombination sites named attL, attB, attP and attR (WO96/40714). Each of these recombination sites or recombination cassettes comprises a pair of recombination sequences (attL1, attL2, attB1, attB2, attP1, attP2 and attR1 and atttR2) and those pairs are flanking the DNA segments to be cloned or to be replaced. These sequences are targets for specific recombination enzymes. The recombination reactions are equivalent to concerted, highly specific cutting and ligation reactions.

The att cloning system and the currently used destination vectors or parts thereof are originally designed to clone one DNA segment. In the Gateway™ system, no destination vectors are provided, which contain two recombination sites with identical pairs of recombination sequences (for example two identical Gateway™ cassettes or Gateway™ cassettes which have the same pair of recombination sequences). A destination vector, which allows the simultaneous cloning of two DNA segments in two Gateway™ cassettes without jeopardizing the fixed cloning orientation and the fixed cloning position of the DNA segments between the recombination sequences of the same recombination site, would overcome the limitations of the existing destination vectors.

Another site-specific recombination system is the Cre/Lox system from the bacteriophage P1 (Stenberg et al., J. Mol. Biol. 187: 197-212, 1986). One limitation of the set of the wild-type Lox recombination recognition sequences is that the recombination reaction is reversible and is shifted in the favor of excision of DNA over insertion of DNA. To overcome this limitation, a system was designed, that favors the insertion process and that also allows for the insertion of multiple segments of DNA into a single vector by successive rounds of recombination (WO0111058). The system uses a vector with different mutated lox sites, that are not able to recombine with each other, but that are able to recombine with a corresponding mutated lox site on another vector and by that recombination, the used recombination sites become inactive. The purpose of this system is to introduce into the same vector multiple DNA segments during successive rounds of recombination.

The system described in WO0111058 however is not designed for simultaneous introduction of two DNA segments at different places in a vector. Furthermore, the system requires different pairs of specially designed, mutated lox site In the destination vector which necessitate the separate preparation of each insert DNA to flank it with the right compatible recombination sequences.

A cloning method for the single-step construction of an expression vector with multiple inserts at defined places and in defined orientation in the vector would overcome the limitations of existing methods.

### SUMMARY OF THE INVENTION

In the present invention novel cloning vectors are designed by using two recombination sites that have the same pair of recombination sequences, and each of those recombination sites allows the insertion/exchange of a DNA segment in a fixed orientation. Said two recombination sites are placed in the same vector but In an opposite orientation.

Unexpectedly, the inventors discovered that when using such a vector with two recombination sites in opposite direction, they could simultaneously introduce two DNA segments in the vector in a single-step recombination reaction. The efficiency of cloning two DNA segments in one vector in this manner was very high and the inserted DNA segments were exactly in the orientation and in the position that was intended. This novel technique solves the problem of cloning two DNA segments in one single site-specific recombination step. Besides the simplicity of making the expression vectors as described in the present invention and the performance of the recombination reaction, the uniformity in preparation of DNA inserts makes this novel cloning technique very attractive for a broad range of applications. One DNA Insert (or entry vector with the DNA insert) can be used twice for the cloning of the DNA insert at two places in the same vector. Also the same DNA insert (or entry vector) can be used to clone the fragment in the different destination vectors.

The construction of co-suppression vectors (for example as described in WO99/53050) by classical cloning techniques is cumbersome, because several rounds of subcloning might be required. The technique of the present invention providing a vector with two recombination sites in opposite directions, allows the cloning of a DNA segment flanked with suitable recombination sequences twice in the same vector In a co-suppression setup. The ability of using the same prepared DNA segment twice and obtaining a co-suppression vector in a single reaction-step is clearly distinct from the existing cloning methods and is extremely useful for efficient and high throughput cloning of co-suppression vectors.

Analogously, the method of the present invention allows the single-step construction of a gene-silencing vector wherein two promoters are cloned in opposite orientation and in between there is a coding region, e.g. the spacer sequences is a coding region. Also a related aspect of the invention is the construction of a polycistronic RNA vector, wherein the two recombination cassettes are separated by an ribosome binding site, so the spacer sequences is an RBS.

The technique of the present invention also allows the cloning of the twice the same or two different DNA segments in for example gene combination vectors so the spacer sequence has two promoters in opposite direction or a bidirectional promoter, promoter combination vectors, wherein the spacer has an Insulator function or gene-promoter combination vectors, wherein the spacer sequences allows efficient transcription and/or translation and is e.g. a Kozak sequence or a RBS. Even more, the technique of the present invention is specially designed for the high throughput cloning of such vectors. The ability of the destination vectors for the uptake of two DNA segments, by cloning two recombination sites in opposite direction, and the uniformity of preparing the DNA segments that have to be introduced make this technique very efficient and can therefore contribute to many industrial applications as well as research projects. The double cloning in one reaction vessel contributes to the saving of reaction components, particularly the expensive recombinase proteins. This feature of the invention is especially important and beneficial for high throughput cloning applications.

Accordingly, the invention relates to a novel cloning vector which comprises two recombination sites which can not interfere with each other, for instance by placing them in the opposite direction. The use of such a vector according to the method of the present invention allows the cloning of the same DNA segments twice or two different DNA segments in one single recombination step. Any recombination system of which the recombination sequences of one recombination sites are not interfering with the recombination sequences of another recombination site, by putting the recombination sites in opposite directions, are suitable for constructing the vectors of the invention and are within the scope of the invention.

The present invention also relates to the production of transgenic cells or organisms comprising the vectors of the present invention. In a preferred embodiment of the invention the transgenic hosts, comprising the DNA molecules described in the present invention are plants. The vectors therefore include plant-operable promoters and 3′ non-translated DNA sequences that function in the cell to cause the addition of polyadenylated nucleotides to the 3′ end of the RNA sequence. In addition to these basic genetic elements of a typical plant expression vector, also other elements for replication, T-DNA transfer, and regulatory sequences can be included. The vector can be constructed in vitro and is subsequently introduced into a plant cell by standard methods to produce a transformed plant cell, and the transformed plant cell is regenerated or grown into a transgenic plant.

Evidently, the method of the present invention can be used for the construction of vectors intended not only for plant transformation, but for any given, species. For combinations of gene cassettes, this would imply use of appropriate regulatory sequences (promoters, terminators) that functions in said species.

### DETAILED DESCRIPTION OF THE INVENTION

The present invention relates more particularly to a DNA molecule for the single-step cloning of two DNA segments, said molecule comprising two first site-specific recombination sites which are

(a) each comprising the same pair of recombination sequences,

(b) each functional for uptake or exchange of a DNA segment,

(c) each reactive to the same second site-specific recombination site, and,

(d) targeted by the same recombinase proteins or recombinase mix,

and wherein the recombination sequences from each of the first recombination sites can recombine with the recombination sequences of the second recombination site, without interference of the recombination sequences of the other first recombination site.

Besides recombination systems such as Gateway™, wherein the sequences of the recombination site have a fixed orientation that determines the orientation of the inserted DNA segment, also non-directional systems, such as Cre/lox, Flp/frt, R/Rs or any other functional equivalents can be used. A preferred way of using multiple of these site-specific recombination sites involves a pair of mutated recombination sites, which do not allow interference of the sequences of the first site with the sequences of the second site. For the Cre/lox system, and to a lesser extent also for the R/RS system, mutated sites have been described that allow recombination with wild-type sites, but not with other mutated sites. In this way, recombination can be allowed between each mutated site in the destination vector with the proper recombination site in the entry. Another example of a modern recombination system which retains the insert's orientation is the Creator™ Gene cloning system (Clontech, Palo Alto, Calif.).

Said DNA molecules of the present invention for instance cover two Identical mutated LoxP sites/pairs that, because of their mutation are no longer able to interfere with each others sequences in the same vector, but that are perfectly able to recombine with the wild-type LoxP or other mutated LoxP sites in another vector.

The meaning of interference is hereunder further clarified by the example of the Gateway™ sequences in the Gateway™ cassettes (herein also referred to as Gateway™ recombination sites).

The attR1 and attR2 recombination sequences of the one of the first recombination sites (which is in the attR1→attR2 orientation) will be together involved in the recombination reaction with the DNA segment flanked by attL1 and attL2, whereby attR1 can only react with attL1 and attR2 with attL2. In this reaction the attR1 and attR2 sequences of the other of the first recombination sites (which is in the attR2→attR1) are involved. However, the attR1 and attR2 sequences of the other of the first recombination sites (which is in the attR2→attR1) can be recombined In a separate recombination event with the insert DNA flanked by the attL1 and attL2 site, resulting in the insertion of the DNA segment in an inverted orientation in the destination vector. This means that the sequences of the recombination sites in the same vector do not recombine with each other. The recombination sequences of an advanced recombination site are made non-cross reactive, which means that the sequences of the same recombination site cannot recombine with each other.

The present invention also relates to a DNA molecule for the single-step cloning of two DNA segments, said molecule comprising two first site-specific recombination sites which are

(a) each functional for uptake or exchange of a DNA segment,

(b) each reactive to the same second site-specific recombination site, and,

(c) targeted by the same recombinase proteins or recombinase mix,

and wherein the recombination sequences from each of the first recombination sites can recombine with the recombination sequences of the second recombination site, without interference of the recombination sequences of the other first recombination site in view of the fact that the two first recombination sites are placed in opposite orientations in said vector.

This is a variation on the afore-mentioned DNA molecule of the invention wherein the recombination sites are not identical, and which are able to interfere when placed twice in the same orientation, but that are made non-interfering by their opposite orientation. There are recombination systems that can be made on-interfering by opposite orientation only when they are adapted/modified (by e.g. Mutations). Lox sequences can for instance be mutated in such a way that when placed in opposite direction with respect to another Lox site, they are unable to interfere with the sequences of the other recombination site in the same vector, but that they are still able to recombine with wild-type Lox sequences of a site in another vector (or with other mutated Lox sequences).

The present invention also relates to a DNA molecule for the single-step cloning of two DNA segments, said molecule comprising two first site-specific recombination sites that are

(a) each comprising the same pair of recombination sequences,

(b) each functional for uptake or exchange of a DNA segment,

(c) each reactive to the same second site-specific recombination site, and,

(d) targeted by the same recombinase proteins or recombinase mix,

and wherein the recombination sequences from each of the first recombination sites can recombine with the recombination sequences of the second recombination site, without interference of the recombination sequences of the other first recombination site in view of the fact that the two first recombination sites are placed in opposite orientations in said vector.

The present invention also relates to any of the DNA molecules of the invention or as defined above wherein the first recombination sequence of said first site-specific recombination sites can only recombine with the first recombination sequence of said second recombination site and wherein the second recombination sequence of said first recombination site can only recombine with the second recombination sequence of said second recombination site.

In this embodiment of the invention recombination sites are used which are directional. The term “directional” in the context of “a directional site-specific recombination site contains a first and a second recombination sequence (that can be either different in composition or in orientation and that allow recombination only with respectively a first and a second recombination sequence of a second recombination site” means that the first sequences of both sites can recombine and that only the second sequences of both sites can recombine. By this, the DNA segment that is cloned will always be cloned in the same orientation. There is no possibility for cross-reactivity between sequence 1 from site 1 and sequence 2 from site 2, or between sequence 2 from site 1 and sequence 1 from site 2. Obviously there is no possibility for recombination between the two sequences of the same site, which is also referred to as cross-reactivity.

An example of a directional recombination site is the Creator™ recombination site or a Gateway™ recombination cassette.

The present invention also relates to any of the DNA molecules of the invention or as defined above wherein said two first site-specific recombination sites comprise att recombination sequences.

The present invention also relates to any of the DNA molecule of the invention or as defined above wherein said two first site-specific recombination sites comprise attR1 and attR2 recombination sequences.

With the expression “attR1 and attR2” is meant two different att R recombination sequences. In a particular embodiment of the Invention, the two different attR sites are the attR1 and attR2 sites of the Gateway™ system.

The present invention also relates to a DNA molecule of the invention as defined above wherein said two first site-specific recombination sites are Gateway™ cassettes, from which a part of the sequence between the recombination sequences has been removed or added or altered.

In the shortened Gateway™ cassette, the negative selection genes (any suicide gene like ccdB) are preferably Intact, while for example the chloramphenicol resistance gene can have been removed. However any part of the Gateway™ cassette between the recombination sites can be removed as long as this deletion does not abolish the recombination capacity of the site. Keeping the suicide gene intact is preferred for the ease of selecting the right construct afterwards. A typical Gateway™ cassette in a destination vector comprises two site-specific recombination sites flanking a selectable marker and/or a suicide gene. Gateway™ cassettes are known in the art and are for instance reviewed in the brochure “Gateway™ cloning technology, featuring Gibco BRL® Products” of the company Ufe Technologies Ltd, Paisley, UK; Walhout A J et al. GATEWAY recombinational cloning: application to the cloning of large numbers of open reading frames or ORFeomes. Methods Enzymol 2000;328:575-92.

The invention further relates to any of the DNA molecules of the invention or as defined above wherein said two first site-specific recombination sites are on the borders of a recombination cassette.

A recombination cassette typically comprises on its borders recombination sequences. The expression “recombination site” also refers to a DNA segment with at its borders two recombination sequences, with whatever sequences there are in between. In a recombination cassette, typically there are other sequences in between the recombination sequences, which allow the easy of cloning and/or the ease selection procedure of the cloned fragments. For example a recombination cassette can comprise a suicide gene that is functional in bacterial cells (e.g. ccdb gene or barnase gene) or another selection gene, which allow the recognition of clones that have the correct cloned DNA segments. For example a typical recombination cassette that can be used in the present invention is a Gateway™ cassette. Also in a particular embodiment of the present invention the Gateway cassette is modified so that a part of the sequence between the recombination sequences has been removed, added or altered.

In a more particular embodiment of the present invention the Gateway cassette Is shortened by removal of a part of the Chloramphenicol selection marker, for instance as explained in Example 1. The part of the Chloramphenicol selection marker, which is removed, may range from at least 10% to about 90% of the total length or any length in-between 10% and 90%.

The present invention therefore also relates to any of the DNA molecules of the invention or as defined above wherein said two first site-specific recombination sites are shortened Gateway™ cassettes, from which a part of the Chloramphenicol resistance gene has been removed.

The present invention also relates to any of the DNA molecules of the invention or as defined above wherein said second recombination site comprises att recombination sequences.

The present invention also relates to a DNA molecule of the invention as defined above wherein said second recombination site comprises attL1 and attL2 recombination sequences.

With the expression “attL1 and attL2” is meant two different att L recombination sequences. In a particular embodiment of the invention, the two different attL sites are the attL1 and attL2 sites of the Gateway™ system.

The present invention also relates to any of the DNA molecules of the invention or as defined above wherein said recombination proteins or said recombination mix is a Clonase™ mix, comprising an integrase, an excisionase and a host factor.

The Clonase™ mix is described in the Gateway system. Typically the clonase mix comprises an integrase, and/or an excisionase and/or a host factor. Examples of clonase mixes that can be used in the present invention are the BP clonase and the LR clonase mix.

In plant genetic engineering the goal Is to produce plants with agronomically Important characteristic traits. Through the technology of plant transformation researchers are able to introduce foreign genes into a plant and thus conferring the desired trait or the quality of interest to the plant.

Therefore, the present invention also relates to any of the DNA molecules of the invention or as described above further comprising elements able to operate with the DNA segment to be cloned and which are important for the effect of said cloned DNA segments in a host cell. It should be clear that the expression “able to operate” as used herein means that once a DNA segment is cloned in said DNA molecule, said element(s) become “operably linked” to said DNA segment.

The “elements” able to operate with the DNA segments to be cloned and which are important for the effect of said cloned DNA segments in the host cell may be of different kinds. The effect of the cloned DNA can be the expression of a protein, expression of RNA or ribozymes. Therefore, elements which are important for this effect can be promoters or other transcriptional regulatory elements, as well as translation regulatory elements, terminators, Kozak sequence, start codon, stop codon etc.

Furthermore, the effect of the cloned DNA segments can be merely a structural effect in the host cell: e.g. interruption of the chromatin structure when inserted or prevent DNA methylation of the host cell DNA. Another effect is for instance tagging the host cell with a particular piece of DNA: the DNA will than only have the function of labelling the host organism (e.g. with PCR one can identify the origin of, or trace a particular organism). Another effect, which does not involve expression of the cloned DNA segments in the host cell is introducing in the host cells regulatory elements: e.g. promoters or enhancer. These can than drive endogenous sequences already present tin the host cell. The elements important for the effect of the cloned segment in the host cells can also be elements that allow the efficient and or stable introduction of the DNA construct of the present invention into the host cell. In a particular embodiment of the present invention, the DNA molecules are equipped with T-DNA borders, which are used to stably transform plant cells and which transfer the DNA molecule of the present invention into the genome of the plant host cell. Other examples of such elements are (but are not limited to) recombination sites that allow homologous recombination.

Accordingly, the invention thus also relates to any of the DNA molecules as defined above wherein said elements are T-DNA borders.

The DNA molecules of the present invention as mentioned above, comprise always two separate first recombination sites which can be used for the single step cloning of two DNA segments. The sequence that is in between the two first recombination sites can vary, as mentioned above, according to the application for which the DNA molecules of the present invention are used. Also, the sequence which is between the first recombination sites (also herein referred to as “intervening sequence” or “spacer sequence”) can have merely a spacer function or alternatively, there is no intervening sequence and the two first recombination sites are adjacent. Accordingly, the intervening sequence can range from 0 base pairs to a larger DNA fragment.

Recent advances in plant molecular biology have shown that a desired characteristic can also be obtained by the silencing of the plant's own gene(s) (Meyer and Saedler (1996) Annu Rev Plant Physiol Plant Mol Biol 47: 23-48; Depicker and Van Montagu (1997) Curr Opin Cell Biol 9: 373-382; Stam et al (1997) Ann Bot 79: 3-12; Gelvin (1998) Curr Opin Biotechnol 9: 227-232; Matzke and Matzke (1998) Curr Opin Plant Biol 1: 142-148; Vaucheret et al (1998) Plant J 16: 651-659; Kooter et al (1999) Trends Plant Sci 4: 340-347; Waterhouse et al (1999) Trends Plant Sci 4: 452-457). As the process of gene silencing in general is being unravelled (Chandler and Vaucheret, (2001), Plant physiology 125: 145-148; Fire (1999) RNA triggered gene silencing. Trends Genet 15: 358), so-called co-suppression vectors are being developed to achieve the silencing of a gene. A typical co-suppression vector contains DNA sequences that, upon transcription are able to form a dsRNA structure, which can be a hairpin. This hairpin is blocking the mRNA for further functionality and will be degraded (WO99/53050, WO99161632, Waterhouse P M, et al. Virus resistance and gene silencing in plants can be induced by simultaneous expression of sense and antisense RNA Proc Natl Acad Sci USA. 1998 Nov. 10;95(23):13959-64, Smith N A, et al. Total silencing by intron-spliced hairpin RNAs, Nature. 2000 Sep. 21;407(6802):319-20.). This aberrant double stranded mRNA structure (e.g. a hairpin) triggers its own destruction, during which also mRNA with sequence homology are eradicated (Montgomery and Fire (1998) Double stranded RNA as a mediator in sequence specific genetic silencing and co-suppression. Trends Genet 14: 255-258; Sharp (1999) RNAi and double stranded RNA. Genes Dev 13: 139-141; Sharp, PA (2001) RNA interference. Genes Dev 15: 535-553). Not only in plants, but also in other organisms gene silencing seems to play a regulating role in cellular mechanisms (Bahramian and Zarbl (1999) Transcriptional and post-transcriptional silencing of rodent alpha (I) collagen by a homologous transcriptionally self-silenced transgene. Mol Cell Biol 19: 274-283; 363Fire et al (1998) Potent and specific genetic interference by double stranded RNA in Caenorhabditis elegans. Nature 391: 806-811; Kennerdell and Carthew (1998) Use of dsRNA-mediated genetic interference to demonstrate that frizzled and frizzled 2 act in the wingless pathway. Cell 95: 1017-1026; Romano and Macino (1992) Quelling: transient inactivation of gene expression in Neurospora crassa by transformation of homologous sequences. Mol Microbiol 6: 3343-3353; Ruiz et al (1998) Homology-dependent gene silencing in Paramecium Mol Biol Cell 9: 931-943). Therefore co-suppression vectors can also be useful for the interference with gene activity of organisms other that plants.

The invention further relates to any of the DNA molecules as described above for the single step construction of a vector wherein said two first site-specific recombination sites are separated (or connected) by a spacer sequence.

The present invention also relates to any of the DNA molecule as defined in the invention wherein the spacer sequence is a DNA sequence of 1 or 3 up to 5000 base pairs.

More particularly, this spacer sequence may have a length of about 5000, 4000, 3000, 2000, 1000, 300, 100 or 10 base pairs.

The present invention also relates to any of the DNA molecules of the invention or as defined above for the single step cloning of a co-suppression vector wherein said two first site-specific recombination sites are connected via a spacer sequence and are both under the control of the same expression control elements (FIG. 4 and example 1 to 4).

For a co-suppression vector the spacer region can be any DNA fragment that serves as a spacer for separating the two flanking DNA regions apart. This spacer region can vary in length, as long as the sense and the antisense flanking sequences are able to form a hairpin. A hairpin refers to any self-annealing double stranded RNA molecule. In its simplest representation, a hairpin RNA consists of a double stranded stem made up by the annealing RNA strands, connected by a single stranded RNA, wherein the single stranded RNA molecule is derived from the spacer region in the DNA molecule. The spacer sequence is also transcribed and it contains no transcription termination sequences, so the downstream sequences can also be transcribed. Preferably this spacer DNA sequences has no internal complementary sequences, so that the spacer region itself (when transcribed) cannot from a hairpin. It can function, (when transcribed) as an RNA loop between the flanking RNA regions. Most preferably this spacer region is a matrix-associated region (MAR sequence). General references for MARS (or SARS) are Cockerill and Garrard (1986) Cell 44: 273-282; Gasser and Laemmli (1986) EMBO J. 5: 511-518; Mirkovitch et al (1986) J Mol Biol 190: 255-258).

The present Invention also relates to any of the DNA molecules of the invention or as defined above wherein said spacer has an insulating function.

For some applications, it might be useful to create sufficient space between the two cloned DNA segments, in order to have an isolation-effect in between the two fragments. This insulating function can be obtained by using a very large spacer or using a particular DNA sequence of which it is known that it has an insulating function, such as a MAR sequence.

Therefore, the present invention also relates to any of the DNA molecules of the invention or as defined above wherein the spacer sequence comprises a MAR sequence.

The MAR sequence to be used in the constructs of the present invention is for instance Nicotiana tabacum matrix associated region.

The present invention thus also relates to any of the DNA molecules of the invention or as defined above wherein the spacer sequence comprises a Nicotiana tabacum MAR sequence.

Alternatives for MARs spacer region are for example a 1022 bp fragment of the GUS gene that was already used by Chuang and Meyerowitz ((2000) PNAS 97: 4985-4990) or the 263 bp spacer with a soybean promoter of the alfa′ subunit of beta-conglycinin (Mette et al (2000) EMBO J. 19: 5194-5201). Also an intron has been used as a spacer region.

The present invention also relates to any of the DNA molecules of the invention or as defined above wherein the spacer sequence comprises a GUS gene sequence, part of the sequence of the soybean promoter of the alpha′ subunit of beta-conglycinin or an intron.

As mentioned above, the spacer can contain different DNA sequences according to the function of the DNA molecule of the present invention. When the DNA molecule is used to construct a co-expression construct, the spacer can consist or can comprise any sequence, but preferably the sequence Is not having a corresponding sequence in the host cell. Also in some cases it is advantageous to have sequences in the spacer region that are not normally transcribed. For example one could use a synthetic piece of DNA, an intron, a polylinklinker of a vector etc.

In analogy to the above mentioned co-suppression vector, wherein the two recombination sites are separated with a spacer sequence and both under the control of the same promoter, the recombination sites can be separated by a coding sequence and are not under the control of a promoter. In this type of vector two promoters can be cloned in opposite directions in the opposite recombination sites, and the promoters can each transcribe the coding sequence (example 8, FIG. 12). This construct can be used for the transcription of that gene in the sense direction and for the transcription of that gene in the antisense direction, generating two transcripts from the same vector, which can form double stranded RNA. This double stranded DNA can be used to trigger gene silencing.

The present invention therefore also relates to a DNA molecule of the invention as defined above wherein the spacer sequence comprises a coding sequence.

The present invention also relates to any of the DNA molecules of the invention or as defined above wherein the spacer sequence allows efficient transcription and or translation of the segments to be cloned.

The present invention also relates to any of the DNA molecules of the invention or as defined above wherein the spacer sequence comprises transcription initiation and/or transcription regulation elements.

The present invention also relates to any of the DNA molecules herein described wherein the spacer sequence comprises 2 separate promoters which can be identical or different, or wherein the spacer sequence comprises a biderectional promoter. Optionally, the distal regions next to the recombination sites In said DNA molecules comprise a terminator sequence. As described in FIG. 9 these construct are used to clone two DNA segments of interest and to put them under separate transcriptional control elements.

Further, the present invention also relates to any of the DNA molecules herein described wherein the spacer sequence comprises 2 separate terminators which can be identical or different, or wherein the spacer sequence comprises a biderectional terminator. Optionally, the distal regions next to the recombination sites in said DNA molecules comprise a promoter sequence. Also in another embodiment of the present invention, the recombination sites can be separated by a ribosome bindings site and can be under the control of the same promoter in order to obtain a multicistronic construct. In this type of vector two genes are cloned in the same orientation by preparing one of those genes in the entry clone in the opposite orientation compared to the orientation of the other gene in the entry clone. When these two entry clones are recombined with the vector of the present invention, the two genes will be in the same direction because the recombination sites in the destination vector are in opposite directions (Example 9, FIG. 13). This type of vector can be used for the simultaneous expression of two genes under the control of the same promoter and for the translation of both genes from the same transcript. The genes that have to be cloned in the polycistronic RNA construct can be the same or different.

Also the desired trait of a transgenic plant can be a result of the combination of more than one introduced gene. Conventional strategies for Introducing multiple genes into target plants are time-consuming and labour intensive. For example, classical breeding methods are used to combine the particular traits by crossing parental plants. Via transgenic procedures, two separate genes can be Introduced in the same plant via simultaneous cotransformation of two vectors, each carrying one of the genes, or by sequential supertransformation of plant comprising a first vector with a first gene, with a second vector, carrying the second gene. Also two transgenic plants can be crossed to obtain combinatorial expression of two transgenes. Another, more sophisticated method for combinatorial expression of two genes, is the use of an internal ribosome binding site in the gene (IRES) from which the second gene can be translated (Wagstaff M J et al. (1998) Gene transfer using a disabled herpes virus vector containing the EMCV IRES allows multiple gene expression in vitro and in vivo. Gene Ther Nov;5(11):1566-70). Still another methodology is based on the construction of a polycistronic operon, wherein the long mRNA encodes multiple genes, separated by translation initiation sites. These two methods involve only one plasmid and consequently only one transformation event, but their limitation lies in the fact that the combined genes are always under the same expression control elements.

Therefore, the present invention also relates to a DNA molecule of the invention as defined above wherein the spacer sequence comprises ribosome binding site.

Finally a vector with a double expression cassette, each comprising a promoter a gene and a terminator can be made by classical cloning methods. As described above, for the construction of these complex vectors, multiple rounds of cloning might be required. Therefore, a single step cloning reaction to introduce two different genes or DNA segments in two different expression cassettes in one vector would constitute an improvement over the existing methods for cloning multiple gene expression vectors. The alternatives for cloning two genes in one single step according to the present inventions are: (1) the same promoters already in the destination vector and cloning the same gene twice behind both promoters for example for having higher expression levels of the gene (2) the same promoters already in the destination vector and cloning two different genes behind the promoters in order to have a gene combination vector for the same expression control of two different genes (3) different promoters already in the destination vector and cloning the same gene twice behind both promoters for having different promoter combinations for the same gene, (4) different promoters already in the destination vector and cloning different genes behind the promoters for having two different promoter-gene combinations. This type of vector is called herein a gene combination vector (Example 5, FIG. 9). The different or the same promoters as referred to above can be in the format of a bidirectional promoter as also illustrated in FIG. 9.

Accordingly, the present Invention also relates to a DNA molecule of the invention as defined above for the single-step cloning of a DNA sequence of interest, particularly a gene, in each of said first site-specific recombination sites, wherein said two first site specific recombination sites are each under the control of separate expression control elements, particularly a promoter.

More particularly, the two sites are under the control of the same promoters or different promoters.

More particularly, the DNA sequence of interest to be cloned in these sites can be the same or can be different.

In a particular application of the present invention, the DNA sequence of interest can be a multigenic DNA fragment, leading to the stacking of genes in each of the two first recombination sites. When these multigenic fragments also contain recombination sites (e.g. according to the present invention, but not interfering with the first set of recombination sites in the vector), it is possible to stack genes or other DNA sequences of interest by multiple rounds of recombination. This is illustrated in FIG. 14.

In another application of molecular biology, the aim is to find the ideal promoter for optimal expression of a certain gene. It would be very useful for the person skilled in the art to have an efficient cloning technique available, for cloning several promoters in front of the genes of interest in one vector. Subsequently this promoter combination vector can be transformed in the host cells and the result of the different expression modules can be evaluated. For plant molecular biology purposes, this kind of vector is very useful for the simultaneous expression of genes in two different tissues of the plant. This can be obtained by cloning for example a root-specific promoter and a seed-specific promoter in front of the gene X, which was already in duplicate in the destination vector. The alternatives for cloning two promoters in one single step according to the present inventions are: (1) the same genes already in the destination vector and cloning the same promoter twice in front of those genes for example for having higher expression of the gene, (2) the same genes already in the destination vector and cloning two different promoters in front of the genes in order to have two expression modules for one gene on the same vector, (3) different genes already in the destination vector and cloning the same promoter twice in front of both genes for the having equal expression control of two different genes, (4) different genes already in the destination vector and cloning different promoters behind the different genes for having two different promoter-gene combinations. This type of vector is called herein a promoter combination vector (example 6, FIG. 10)

The present invention also relates to a DNA molecule of the invention as defined above for the single step cloning of a expression control element, particularly a promoter, in each of said first site-specific recombination sites, wherein said two first recombination sites are each followed by a DNA sequence of interest, preferably a gene.

More particularly, the two sites are followed by the same gene or different genes.

The present invention also relates to a DNA molecule of the invention as defined above wherein the spacer sequence is an insulating sequence and wherein the DNA molecule further comprises at least one coding sequence next to a first recombination.

More particularly, the promoters to be cloned in these sites can be the same or can be different.

More particularly, “followed” means that at a reasonable distance downstream the DNA sequence of interest starts. In a particular case the promoter is followed first by an 5′ untranslated region or another intervening sequence and than by the DNA sequence of interest.

Alternatively, when a promoter has to be combined with gene, it is very useful to be able to do the cloning in one single reaction step. Currently this construction involves multiple rounds of cloning (restriction, isolation, ligation). Even more, when this procedure has to be done regularly with a whole set of promoters and a set of genes, it is important to have standardized procedures which are the same for every sequence. According to a vector of the present invention wherein two recombinations sites are adjacent but in opposite directions, one is able to clone a promoter and a gene in a single step. For high throughput cloning this technique is especially useful since the whole set of promoters and the whole set of genes can be prepared the same way by flanking with the proper recombination sites. However for the preparation of the inserts, for example the promoter set in the entry clone, the promoter in the entry clone has to be in the opposite orientation compared to the orientation of the gene in the entry clone. Upon recombination of the promoter from that entry clone and the gene from that entry clone in the destination vector, the promoter and the gene will be in the same orientation, because the recombination sites in the destination vector are in opposite directions. Only one, or both of the two recombination sites in this type of vector can be flanked by a 3′ UTR's. This type of vector is called herein a promoter-gene combination vector (example 8, FIG. 11).

The present invention thus also relates to a DNA molecule of the invention as defined above for the single-step cloning of an expression control element, particularly a promoter, in one of the first site-specific recombination site and a DNA sequence of interest, particularly a gene, in the other first site-specific recombination site, wherein said first recombination sites are adjacent to each other.

More particularly adjacent means in a reasonable distance from each other. In a particular case the DNA sequence connecting the adjacent recombination sites can vary in length (it can for example comprise a 5′UTR or a ribosome binding site or other translation initiation sequences) and in composition of sequence (as long as it does not hamper the control of for example the promoter on the gene).

Also in the area of molecular biology, companies as well as academic research groups are more and more involved in large-scale projects, wherein whole libraries of different DNA sequences have to be screened and analyzed for the desired effect. In many cases the initial procedures of these high-throughput processes, require the cloning of the DNA sequences in the suitable vector. To work efficiently and reduce the consumption of time and money, it is extremely important for the person skilled in the art to make use of a standardized method, allowing the cloning of all the DNA fragments in the same way, independently from the length or the composition of the DNA segment. For this reason it is beneficial to work with site-specific recombination. Furthermore, a site-specific recombination based technology that also allows the high throughput cloning of vectors with multiple inserts at multiple sites in the vector would constitute an improvement over other techniques currently used for the development of co-suppression vectors, gene combination vectors, promoter combination vectors or promoter-gene combination vectors.

According to a further embodiment the invention relates to any of the DNA molecules as herein described for the high throughput cloning of two copies of the same DNA segment.

The present invention also relates to a DNA molecule of the invention as defined above for the high throughput cloning of twice the same DNA segment in one vector but in said two first site-specific recombination sites, wherein said DNA segment prior to recombination is flanked by the sequences of said second recombination site.

Further the invention also relates to any of the DNA molecules as herein described for the high throughput cloning of two different DNA segments.

The present invention also relates to a DNA molecule of the invention as defined above for the high throughput cloning of two different DNA segments in one vector but in said two first site-specific recombination sites, wherein the two different DNA segments prior to recombination are flanked by the sequences of said second recombination site.

The present invention also relates to any of the DNA molecules of the invention or any of the DNA molecules as defined above wherein the expression control elements are functional in prokaryotes and eukaryotes.

The present invention also relates to any of the DNA molecules of the invention or any of the DNA molecules wherein the expression control elements are functional in plants.

The invention also relates to any of the DNA molecules as described herein wherein the first two recombination sites are comprised within T-DNA borders.

The invention further relates to the use of any of the DNA molecules of the invention for the single step cloning of two different DNA segments.

The invention also relates to the use of any of the DNA molecules of the invention for the single step cloning of two identical DNA segments.

The invention further relates to the use of any of the DNA molecules of the invention for the single step construction of a co-suppression vector

The invention further relates to the use of any of the DNA molecules of the invention for the single step construction of a co-suppression vector for plants, wherein the two first recombination sites are comprised within T-DNA borders.

Alternatively, the two first recombination sites may be under the control of a promoter, preferably any promoter, other than the T7 promoter, preferable a plant promoter.

In another embodiment of the present invention any of the DNA molecules may further contain a terminator sequence that is operably linked to the co-suppression module built by the two first recombination sites.

According to a further embodiment of the invention, DNA molecule as described above, may contain a co-suppression module and further comprising elements that allow the functionality of the co-suppression module in plants, such as T-DNA borders.

The invention further relates to the use of any of the DNA molecules of the invention for the single-step cloning of two DNA segments of interest, particularly genes or reporter genes, in each of said first site-specific recombination sites, wherein said DNA segments are identical or different, and wherein each of said two first site specific recombination sites is under the control of expression control elements, wherein said expression control elements are identical or different.

The invention further relates to the use of any of the DNA molecules of the invention for the single step cloning of expression control elements, particularly promoters, in each of said first site-specific recombination sites, characterized in that said DNA molecule further comprises upstream or downstream of each of said two first recombination sites, a DNA sequence of interest, particularly a gene or a reporter gene, wherein said DNA sequences are identical or different.

The invention further relates to the use of any of the DNA molecules of the invention for the single-step cloning of an expression control element, particularly a promoter, in one of the first site-specific recombination site and a DNA sequence of interest, particularly a gene or a reporter gene in the other first site-specific recombination site.

The invention further relates to the use of any of the DNA molecules of the invention for the single-step cloning of two expression control elements, particularly promoters, in opposite direction, wherein the two first recombination sites are separated by a coding region.

The invention further relates to the use of any of the DNA molecules of the invention for the single-step cloning of two DNA segments of interest, particularly genes, to generate a polycistronic RNA.

The invention further relates to the use of any of the DNA molecules of the invention for the purpose of gene stacking, by single-step cloning of two DNA segments of interest, wherein each of said DNA segments comprises at least one recombination site which is identical to at least one of said first recombination sites. An example of such a construction is represented in FIG. 14.

The invention further relates to the use of any of the DNA molecules of the invention for the single-step cloning of two DNA segments, particularly transcription control elements, to compose a bidirectional promoter.

For the particular use of the invention as described above, the two first recombination sites are separated by the spacer sequence and the DNA segments to be cloned contain a promoter element. When cloning this promoter element in the opposite direction, the spacer element will function as a linker that joins the two promoter elements in one bidirectional promoter, that is able to drive the expression of genes in the opposite direction (see FIG. 13).

The invention further relates to the use of any of the DNA molecules of the invention for the single-step cloning of two DNA segments of interest, particularly expression modules comprising a gene and a promoter.

These expression modules may be generated by the combigate methodology (European patent application EP 02075373.7). Alternatively, the cloned DNA segments are mutated according to the combigate technology. Therefore the combination of the combigate technology, which allows the flexible construction of an entry clone, with the vectors and the methods of the present invention are extremely efficient for high throughput cloning.

The invention further relates to the use of any of the DNA molecules of the invention for the single-step cloning of two DNA segments of interest for generating one or more fusion proteins.

For this particular use of the DNA molecule of the invention, there are several alternative possibilities. For example, the cloned DNA segments are a promoter and a coding sequence (the first portion of a fusion protein), and the DNA molecule according to the present invention further comprises another gene or functional part thereof (the second portion of a fusion protein) to be included in a fusion protein. This means that one of the first recombination sites is linked to that gene or functional part thereof already present in the vector in such a way that in frame translation is possible between the first and the second portion of the fusion protein.

Alternatively, there are two promoters present in the DNA molecule of the invention, each followed by a fist recombination site, which is followed by the second portion of the fusion protein. These portions can be different so that in a single step two different fusion proteins are made with the same DNA segment to be cloned. Alternatively, the second portions of the fusion proteins are identical, but the two promoters are different, allowing one to two expression modes for the fusion protein.

Alternatively, the two cloned DNA segments are two portions of the fusion protein that will be fused by cloning. In this case, the spacer sequence between the two first recombination sites in the DNA molecule of the invention allows in frame translation of the two portions of the fusion proteins. In one example, this spacer sequence is as small as possible, functions as an adapter to put the two cloned fragments in frame and does not contain a stop codon. In a particular embodiment a DNA segment will be incorporated in a fusion protein with GUS, GFP, or another reporter gene. Also in a particular embodiment of the invention, fusions are made to tag sequences, such as epitope sequences. These tag sequences will allow the easy detection of the DNA or protein whereto it is coupled. Examples of such epitope tags are e.g. E-tag, c-myc tag etc.

As shown in FIG. 18, it is shown that it is also possible that the above-described “second part of the fusion protein” can also be fused N-terminal of the above described “first part of the fusion protein”. In analogy to the C-terminal fusions, there are several possibilities to use the two first recombination sites according to the present invention, in order to create one or more fusion protein. For example, they can be used to choose the promoter and the first part of the fusion, or the promoter in combination with the second part of the fusion, or to choose the two parts of the fusion protein, or to choose only one part of the fusion protein, but then in two copies etc.

The present invention also relates to a method for simultaneous cloning of two DNA segments at two different locations in a single vector during a single-step recombination reaction, comprising the steps of:

(a) generating a destination vector of the Invention as defined above,

(b) generating one or more insert DNA segment(s) flanked with the sequences of said second site-specific recombination site, and,

(c) performing a single-step recombination reaction by combining the destination vector and the insert segment or segments in the presence of the recombinase proteins or recombinase mix.

More particularly, a single-step recombination reaction means that all the necessary recombinations or recombination events are performed in one reaction, possible in one reaction vessel and in one reaction mix. In a more particular embodiment of the invention, a single step recombination reaction means a reaction with the same recombination protein (such as the same clonase TM mix).

The present invention also relates to a method of the invention as defined above for inserting two identical or different copies of a DNA segment for obtaining one of a co-suppression vector, a gene combination vector, a gene silencing vector, a polycistronic (RNA) vector, a promoter combination vector, a promoter-gene combination vector, a gene stacking vector, or a vector comprising a bidirectional promoter or combinatorial expression cassettes, or a fusion vector.

The present invention relates also to a DNA molecule obtainable by a method of the invention as defined above.

The present invention relates also to a transgenic host cell comprising a DNA molecule of the invention as defined above.

The present invention relates also to a transgenic plant, in particular a crop plant, comprising a DNA molecule of the invention as defined above.

The present invention relates also to a transgenic non-human mammalian organism comprising a DNA molecule of the invention as defined above,

The present invention relates also to a transgenic organisms, which can be selected from the group comprising prokaryotes, eukaryotes, animal, fish, insects, yeast, mold, fungi, nematodes etc., comprising the DNA molecules of the invention as defined above.

More particularly, the present invention relates to a transgenic cell or organism, comprising the molecule of the present invention, wherein said DNA molecule resides in the genome of said cell or organism

The advantages of the methods of the present invention can be found in any of the following features: the single step reaction, the saving of the recombinase enzyme, the use of two recombination sites which have the same pair of recombination sequences, the same preparation of the insert DNA segments for double cloning and the compatibility for high throughput cloning. The DNA segments to be cloned can be of any origin or type (even multiple gene fragments).

Further, the inserted DNA segments, when cloned twice from the same entry vectors will have opposite direction, since the recombination sites in the destination vector are in opposite orientation. This is extremely useful for co-suppression vectors, but can also be used for other two-insert vectors. The inserted DNA segments in the expression vector can also have the same orientation when they were cloned from separate entry clones wherein the orientation of the first gene in the first entry clone is opposite to the orientation of the second gene in the second entry clone.

The ease of making a co-suppression construct previously involved a great deal of time (i.e. weeks) and effort is spent on the initial amplification and cloning of the first DNA segment, and subsequently in the amplification and the transfer of the second DNA segment in a inverse orientation the final co-suppression vector. The current approach (and vector) allows the construction of a high number of co-suppression constructs over a short time period.

Definitions and Elaborations to the Embodiments

In the description of the Invention, a number of terms used in recombinant DNA technology are utilized extensively. In order to provide a clear and consistent understanding of the specification and claims, including the scope to be given such terms, the following definitions are provided.

The Gateway™ recombination system (www.lifetech.com/gateway,) is described as: “The site-specific cloning method of Gateway™ comprises several steps using specially designed recombination sites named attL, attB, attP and attR (WO96/40714, U.S. Pat. No. 5,888,732, U.S. Pat. No. 6,143,557) flanking the DNA segments to be cloned or replaced. These recombination sites are mutated from the wild-type λ att sites in order to improve the specificity and the efficiency of the recombination reaction. These mutations involved removal and or substitution of internal sequences and elimination of stop codons. The recombination reaction, is the main pathway of the system, mediated by the LR clonase mix of recombination proteins (int (integrase), IHF (integration host factor), Xis (excisionase)). This reaction transfers DNA segments (cDNA, genomic DNA or gene sequences) of the entry vector to the destination vector, to create an expression vector. The Gateway™ cloning reactions are equivalent to concerted, highly specific cutting and ligation reactions. Viewed in this way, the recombination proteins cut to the left and the right of the gene in the entry vector and ligate it into the destination vector, creating a new expression vector. During this process, the reading frame is maintained as well as the orientation of the insert. The insert gene or insert DNA segment in the entry vector is flanked by attL1 and attL2 sites, while in the destination vector the DNA segments that is to be replaced by the gene is flanked by the attR1 and attR2 sites. After recombination, the gene in the expression vector is flanked by attB1 and attB2 sites. The orientation of the gene is maintained throughout the subcloning because the recombination sequence attL1 reacts only with attR2, and attL2 reacts only with attR2, due to their fixed orientation in the recombination site and their asymmetric base pair constitution.

The recombination (att) sequences of each vector comprise a hybrid sequence, donated by the parental vectors (FIG. 1). This makes the recombination site directional, meaning that the inserted DNA segments can only be inserted in one possible orientation. This structure is shown in detail in FIG. 1 wherein the staggered lines, dividing the two portions of the att sequence, represent the seven-base staggered cut produced by Int during the recombination reaction. This is demonstrated by the display of the recombination sequences of an attB expression vector, generated by recombination between the attL1 and attL2 sites of an entry vector with respectively the attR1 and attR2 sites of a destination vector.”

With “acceptor” or “destination vector” or construct is meant the backbone DNA fragment which will be the recipient for other DNA fragments coming form the donor, entry vector, plasmid or construct. Different types of cloning vectors are available such as plasmids, bacteriophages, artificial chromosomes, etc.

The term “host” refers to any prokaryotic or eukaryotic organism that can be a recipient of the recombinational cloning Product. A “host,” as the term is used herein, includes prokaryotic or eukaryotic organisms that can be genetically engineered. For examples of such hosts, see Maniatis et al., Molecular Cloning. A Laboratory Manual, Cold Spring Harbor Laboratory, Cold Spring Harbor, N.Y. (1982).

The term “insert” refers to the desired DNA segment, which one wishes to clone in a particular vector or other DNA fragment. The insert can contain one or more genes, one or more control elements or other functional or non-functional DNA sequences. In a particular embodiment of the present invention, the inserted DNA segments again contain the recombination sites as described in the present invention, in order to allow a subsequent round of single-step introduction of two DNA segments. This approach allows creating multiple gene inserts, or allows gene-stacking.

The term “entry vector” or “entry clone” refers to the insert donor DNA. The entry clone is one of the two parental DNA molecules of the present Invention, which carries the insert. The entry vector DNA molecule comprises the Insert flanked on both sides with recombination sequences. The entry vector can be linear or circular. In one embodiment of the invention, the insert donor is a circular DNA molecule and further comprises a cloning vector sequence outside of the recombination sequences.

The term “destination vector” refers to the DNA segment, which will take up the insert DNA to be cloned. The destination vector is a cloning vector comprising a circular or linear DNA molecule, which includes an appropriate replicon. In the present invention, the destination vector contains functional and/or regulatory elements that are desired to be incorporated into the final product to act upon or with the cloned DNA Insert. The destination vector can also contain a selectable marker. The destination vector is one of the two parental DNA molecules of the present invention, which carries the DNA segments encoding the DNA molecule which is to become part of the desired product. The cloning technique of the present invention can be used for (1) the cloning of cDNA, genomic DNA, chromosomes, YACs and other artificial chromosomes, PCR fragments, mRNA, etc. in any vector such as the widely known pUC, pGEM, pBluescript, and (2) the subcloning of these fragments into specialized vectors for functional analysis.

The term “product” or “construct” refers to the desired daughter molecules after the recombinational cloning process. The product or construct contains the DNA which was to be cloned or subcloned and contains the parts of the destination vector intended for the further use of the DNA insert.

The term “recognition sequence” refers to particular DNA sequences which a protein, DNA, or RNA molecule (e.g., restriction endonuclease, a modification methylase, or a recombinase) recognizes and binds. For example, the recognition sequence for Cre recombinase is loxP which is a 34 base pair sequence comprised of two 13 base pair inverted repeats (serving as the recombinase binding sequence) flanking an 8 base pair core sequence. See also FIG. 1 of Sauer, B., Current Opinion in Biotechnology 5:521-527 (1994). Other examples of recognition sequences are the attB, attP, attL, and attR sequences which are recognized by the recombinase enzyme 1 Integrase. attB is an approximately 25 base pair sequence containing two 9 base pair core-type Int binding sites and a 7 base pair overlap region. attP is an approximately 240 base pair sequence containing core-type Int binding sites and arm-type Int binding sites as well as sites for auxiliary proteins IHF, FIS, and Xis. See for instance Landy, Current “Opinion in Biotechnology 3:699-707 (1993). Such sites are also engineered according to the description of the Gateway™ system to enhance the cloning methods and the vector products (Gateway (www.lifetech.com/gateway).

The term “recombination sequence” refers in general to a DNA molecule or a part of a DNA molecule of an integrase family site-specific recombination system. A recombination sequence is a sequence which is recognized by a recombinase protein. In a particular example, the term recombination sequence refers to a sequence of a recombination site (e.g. AttR1, AttR2, AttB1, AttB2, AttP1, AttP2, AttL1 and AttL2 of the attR, attB, attP or attL recombination site or of one of the Gateway™ cassettes). Another example is the Lox (34 bp) inverted repeat that is the recombination sequence of the LoxP pair, or the LoxP recombination site). The AttR1, AttR2, AttB1, AttB2, AttP1, AttP2, AttL1 and AttL2 have a fixed orientation and are asymmetric, therefore they determine the fixed orientation of the inserted DNA segment and only one orientation is possible.

The expression “att site” as used herein refers to all possible att recombination sites such as attB, attP, attL, attR.

The term “recombination site” refers to a place where the DNA will be inserted and/or where the DNA will be replaced. The expression “recombination site” also refers to a DNA segment which comprises at its borders recombination sequences, regardless if there are other sequences in between the recombination sequences or not. For example, the att recombination sites are used in the Gateway system and the LoxP pair constitutes the recombination site of the Cre/Lox system).

The term “recombination cassette” refers to a DNA segment that contains recombination sequences and preferably, but not necessarily a selectable marker or a screenable marker.

A recombination cassette typically comprises on its borders recombination sequences. In a recombination cassette, typically there are other sequences in between the recombination sequences, which allow the easy cloning and/or the easy selection of the clones comprising the correct cloned DNA segments. For example, a Gateway™ cassette is a recombination site comprising for example attR1+intermediate sequence with the suicide gene ccdb and with an antibiotic resistance gene+attR2.

The expression “recombinase mix” as used herein means a mixture that comprises at least one recombinase protein. For example the ™Clonase mix of Clontech (e.g. the BP clonase mix or the LR clonase mix) is a recombinase mix that can be used in the methods of the present invention. A typical “recombination mix” includes excisive or integrative proteins, enzymes, co-factors or associated proteins that are involved in recombination reactions involving one or more recombination sites. Site specific recombinases are enzymes that are naturally present in some viruses and bacteria and have been characterized to have both endonuclease and ligase properties. These recombinases (along with associated proteins) recognize specific sequences of bases in DNA and exchange the DNA sequences flanking those segments. The recombinases and associated proteins are collectively referred to as recombination mix (see Landy, A. Current opinion in Genet. Dev. 3: 699-707 (1993)). For the Gateway Gateway™ system, the recombinase process is a cooperative reaction between several proteins namely integrase, excisionase and host factor. Therefore when used herein “recombinase enzyme or recombinase mix” also means a protein mix which consist of proteins that are able to mediate a recombination reaction. Furthermore, with “recombinase enzyme” is also meant any recombinase complex, consisting of several interacting proteins. The term “site-specific recombinase” refers to a type of recombinase which typically has at least the following four activities: (1) recognition of one or two specific DNA sequences; (2) cleavage of said DNA sequence or sequences; (3) DNA topolsomerase activity involved in strand exchange; and (4) DNA ligase activity to reseal the cleaved strands of DNA. See for instance Sauer, B., Current Opinion in Biotechnology 5:521-527 (1994). Conservative site-specific recombination is distinguished from homologous recombination and transposition by a high degree of specificity for both partners. The strand exchange mechanism involves the cleavage and rejoining of specific DNA sequences in the absence of DNA synthesis (Landy, A. (1989) Ann. Rev. Biochem. 58:913-949). Numerous recombination systems from various organisms have been described. Many of these belong to the integrase family of recombinases (Agros et al. EMBO J. 5:433-440 (1986)). Perhaps the best studies of these are the integrase/att system from bacteriophage λ (Landy A. Current opinions in Genet. Devel. 3: 699-707 (1993)), The Cre/loxP system from bacteriophage P1 (see for instance the work of Hoess R and Abremski K e.g. Abremski K, et al. “Bacteriophage P1 Cre-loxP site-specific recombination. Site-specific DNA topoisomerase activity of the Cre recombination protein” J Biol. Chem. 1986 Jan. 5; 261(1):391-6), and the FLP/FRT system from Saccharomyces cerevisiae 2μ circle plasmid (Broach J R, Guarascio V R, Jayaram M. “Recombination within the yeast plasmid 2 mu circle is site-specific” Cell. 1982 May;29(1):227-34).

The term “recombinase” refers to an enzyme, which catalyzes the exchange of DNA segments at specific recombination sites.

The term “recombination reaction” refers to a method described herein, whereby segments of DNA molecules are exchanged, inserted, replaced, substituted or modified, in vitro or in vivo.

“Recombination” generally refers to the joining of nucleic acids. Homologous recombination can occur between DNA sequences that have nucleotide sequences in common. Site-specific recombination refers to recombination that occurs at specific regions that is regulated by specific enzymes known as site-specific recombinases. These site-specific recombinases recognize short stretches of DNA and a cross-over or physical exchange of DNA can take place in the presence of the recombinase. A number of different site specific recombinase systems describe Including the Cre/lox system of bacteriophage P1, the FLP/FRT system of yeast, the Gin recombinase of phage Mu, the Pin recombinase of E. coli, the PinB, PinD and PinF from Shigella, and the R/RS system of Zygosaccharomyces rouxii. Recombinases generally are integrases, resolvases or flippases. The best-studied site-specific recombinase systems are the bacteriophage P1 Cre/lox, the yeast FLP/FRT and the Z. rouxii R/RS systems. In these systems a recombinase (Cre, FLP or R) interact specifically with its respective site-specific recombination sequence (lox, FRT, or RS respectively) to invert or excise the intervening sequences. The site-specific recombination sequences for each of these two systems are relatively short (34 bp for 10× and 47 bp for FRT).

A “site-specific recombination reaction” refers to a method including mixing all components necessary to initiate a recombination event between a destination nucleic acid and a donor nucleic acid which contains the insert DNA fragment, said reaction being characterized in that the exchangeable DNA fragments are flanked by compatible recombination sequences which make part of an recombination sites and that the exchange is mediated under the activity of recombinase proteins. The donor nucleic acid can be a donor plasmid, or a PCR fragment, or even a synthetic DNA fragment (oligo) all with the suited flanking sequences.

Cre/Lox: The Cre recombinase recognizes a 34 base pair lox sequence that contains two 13 base pair recombinase recognition/inverted repeat sequences. Such a lox sequence is also referred to as LoxP. Four molecules of the Cre recombinase will recognize and bind to a pair of Lox sequences (one Cre molecule per inverted repeat sequence) and catalyses a reciprocal crossover between these sequences. If the asymmetric lox sequence is directly repeated the DNA sequence between the lox sites will be excised. If the lox sequences are in an inverted orientation, the DNA sequence between the Lox sequences will be inverted.

Other more general terminology is hereunder further explained in order to define the scope of the terms.

Those skilled in the art will be aware that the invention described herein is subject to variations and modifications other than those specifically described. It is to be understood that the invention described herein includes all such variations and modifications. The invention also includes all such steps, features, compositions and compounds referred to or indicated in this specification, individually or collectively, and any and all combinations of any or more of said steps or features.

Throughout this specification, unless the context requires otherwise the word “comprise”, and variations such as “comprises” and “comprising”, will be understood to Imply the inclusion of a stated integer or step or group of integers or steps but not the exclusion of any other integer or step or group of integers or steps.

As used herein, the term “derived from” shall be taken to indicate that a particular integer or group of integers has originated from the species specified, but has not necessarily been obtained directly from the specified source.

The terms “gene(s)”, “nucleic acid sequence(s)”, “nucleotide sequence(s)”, “DNA sequence(s)” or “DNA molecule” or “nucleic acid molecule(s)”, when used herein refer to nucleotides, either ribonucleotides or deoxyribonucleotides or a combination of both, in a polymeric form of any length. Said terms furthermore include double-stranded and single-stranded DNA and RNA. Said terms also include known nucleotide modifications such as methylation, cyclization and ‘caps’ and substitution of one or more of the naturally occurring nucleotides with an analogue such as inosine. Modifications of nucleotides include the addition of acridine, amine, biotin, cascade blue, cholesterol, Cy3®, Cy5®, Cy5.5® Dabcyl, digoxigenin, dinitrophenyl, Edans, 6-FAM, fluorescein, 3′-glyceryl, HEX, IRD-700, IRD-800, JOE, phosphate psoralen, rhodamine, ROX, thiol (SH), spacers, TAMRA, TET, AMCA-S®, SE, BODIPY®, Marina Blue®, Pacific Blue®, Oregon Green®, Rhodamine Greene®, Rhodamine Red®, Rhodol Green® and Texas Red®. Polynucleotide backbone modifications include methylphosphonate, 2′-OMe-methylphosphonate RNA, phosphorothiorate, RNA, 2′-OMeRNA. Base modifications include 2-amino-dA, 2-aminopurine, 3′-(ddA), 3′dA(cordycepin), 7-deaza-dA, 8-Br-dA, 8-oxo-dA, N⁶-Me-dA, abasic site (dSpacer), biotin dT, 2′-OMe-5Me-C, 2′-OMe-propynyl-C, 3′-(5-Me-dC), 3′-(ddC), 5-Br-dC, 5-1-dC, 5-Me-dC, 5-F-dC, carboxy-dT, convertible dA, convertible dC, convertible dG, convertible dT, convertible dU, 7-deaza-dG, 8-Br-dG, 8-oxo-dG, O⁶-Me-dG, S6-DNP-dG, 4-methyl-indole, 5-nitroindole, 2′-OMe-inosine, 2′-dl, O⁶-phenyl-dl, 4-methyl-indole, 2′-deoxynebularine, 5-nitroindole, 2-aminopurine, dP(purine analogue), dK(pyrimidine analogue), 3-nitropyrrole, 2-thio-dT, 4-thio-dT, biotin-dt, carboxy-dT, O⁴-Me-dT, O⁴-triazol dT, 2′-OMe-propynyl-U, 5-Br-dU, 2′-dU, 5-F-dU, 5-I-dU, I⁴-triazol dU and radiolabels (e.g. ¹²⁵I, ¹³¹I, ³⁵S, ¹⁴C, ³²P, ³³P, ³H). Said terms also encompass peptide nucleic acids (PNAs), a DNA analogue in which the backbone is a pseudopeptide consisting of N-(2-aminoethyl)-glycine units rather than a sugar. PNAs mimic the behaviour of DNA and bind complementary nucleic acid strands. The neutral backbone of PNA results in stronger binding and greater specificity than normally achieved. In addition, the unique chemical, physical and biological properties of PNA have been exploited to produce powerful biomolecular tools, antisense and antigene agents, molecular probes and biosensors. The term “DNA fragment or DNA segment” means any DNA fragment, which is derived or prepared from an original (larger) DNA molecule. These terms are not restrictive to the content of the DNA fragment or segment, which can be any DNA, with any functionality. For example the DNA fragment or segments can comprise one or more genes, control elements like promoters and terminators, or it can contain just spacer sequences etc. Also the DNA fragment can contain recognition sequences, like recombination sequences etc.

With “recombinant DNA molecule” or “chimeric gene” is meant a hybrid DNA produced by joining DNA fragments from different sources. “Sense strand” refers to the strand of a double-stranded DNA molecule that is homologous to a mRNA transcript thereof. The “anti-sense strand” contains an inverted sequence, which is complementary to that of the “sense strand”.

A “coding sequence” or “open reading frame” or “ORF” is defined as a nucleotide sequence that can be transcribed into mRNA and/or translated into a polypeptide when placed under the control of appropriate regulatory sequences, i.e. when said coding sequence or ORF is present in an expressible format. Said coding sequence of ORF is bounded by a 5′ translation start codon and a 3′ translation stop codon. A coding sequence or ORF can include, but is not limited to RNA, mRNA, cDNA, recombinant nucleotide sequences, synthetically manufactured nucleotide sequences or genomic DNA. Said coding sequence or ORF can be interrupted by intervening nucleic acid sequences.

To effect expression of a protein in a cell, tissue or organ, preferably of plant origin, either the protein may be introduced directly to said cell, such as by microinjection or ballistic means or alternatively, an isolated nucleic acid molecule encoding said protein may be introduced into said cell, tissue or organ in an expressible format.

With “vector” or “vector sequence” is meant a DNA sequence, which can be introduced in an organism by transformation and can be stably maintained in said organism. Vector maintenance is possible in e.g. cultures of Escherichia coli, A. tumefaciens, Saccharomyces cerevisiae or Schizosaccharomyces pombe. Other vectors such as phagemids and cosmid vectors can be maintained and multiplied in bacteria and/or viruses. Vector sequences generally comprise a set of unique sites recognised by restriction enzymes, the multiple cloning site (MCS), wherein one or more non-vector sequence(s) can be Inserted. Examples include plasmids, phages, and other DNA sequences which are able to replicate or to be replicated in vitro or in a host cell, or to convey a desired DNA segment to a desired location within a host cell. A vector can have one or more restriction endonuclease recognition sites at which the DNA sequences can be cut in a determinable fashion without loss of an essential biological function of the vector, and into which a DNA fragment can be spliced in order to bring about its replication and cloning. Vectors can further provide primer sites, e.g., for PCR, transcriptional and/or translational initiation and/or regulation sites, recombinational sites, replicons, Selectable markers, etc.

Clearly, methods of inserting a desired DNA fragment which do not require the use of homologous recombination or restriction enzymes (such as, but not limited to, UDG cloning of PCR fragments (U.S. Pat. No. 5,334,575), T:A cloning, and the like) can also be applied to clone a DNA fragment into a cloning vector to be used according to the present invention. The cloning vector can further contain a selectable marker suitable for use in the identification of cells formed with the cloning vector.

“Expression vectors” form a subset of vectors which, by virtue of comprising the appropriate regulatory sequences enabling the creation of an expressible format for the inserted non-vector sequence(s), thus allowing expression of the protein encoded by said non-vector sequence(s). Expression vectors are known in the art enabling protein expression in organisms including bacteria (e.g. E. coli), fungi (e.g. S. cerevisiae, S. pombe, Pichia pastoris), insect cells (e.g. baculoviral expression vectors), animal cells (e.g. COS or CHO cells) and plant cells (e.g. potato virus X-based expression vectors, see e.g. Vance et al. 1998—WO9844097).

By “expressible format” Is meant that the isolated nucleic acid molecule is in a form suitable for being transcribed into mRNA and/or translated to produce a protein, either constitutively or following induction by an intracellular or extracellular signal, such as an environmental stimulus or stress (mitogens, anoxia, hypoxia, temperature, salt, light, dehydration, etc) or a chemical compound such as IPTG (isopropyl-β-D-thiogalactopyranoside) or such as an antibiotic (tetracycline, ampicillin, rifampicin, kanamycin), hormone (e.g. gibberellin, auxin, cytokinin, glucocorticoid, brassinosteroid, ethylene, abscisic acid etc), hormone analogue (iodoacetic acid (IAA), 2,4-D, etc), metal (zinc, copper, iron, etc), or dexamethasone, amongst others. As will be known to those skilled in the art, expression of a functional protein may also require one or more post-translational modifications, such as glycosylation, phosphorylation, dephosphorylation, or one or more protein-protein interactions, amongst others. All such processes are included within the scope of the term “expressible format”.

Preferably, expression of a protein in a specific cell, tissue, or organ, preferably of plant origin, is effected by introducing and expressing an isolated nucleic acid molecule encoding said protein, such as a cDNA molecule, genomic gene, synthetic oligonucleotide molecule, mRNA molecule or open reading frame, to said cell, tissue or organ, wherein said nucleic acid molecule is placed operably in connection with suitable regulatory sequences including a promoter, preferably a plant-expressible promoter, and a terminator sequence.

“Regulatory sequence” or “expression control elements” refers to control DNA sequences, which are necessary to affect the expression of coding sequences to which they are ligated. The nature of such control sequences differs depending upon the host organism. In prokaryotes, control sequences generally include promoters, ribosomal binding sites, and terminators. In eukaryotes generally control sequences include promoters, terminators and enhancers or silencers. The term “control element” is intended to include, at a minimum, all components the presence of which are necessary for expression, and may also include additional advantageous components and which determines when, how much and where a specific gene is expressed.

Reference herein to a “promoter” is to be taken in its broadest context and includes the transcriptional regulatory sequences derived from a classical eukaryotic genomic gene, including the TATA box which is required for accurate transcription Initiation, with or without a CCAAT box sequence and additional regulatory elements (i.e. upstream activating sequences, enhancers and silencers) which alter gene expression in response to developmental and/or external stimuli, or in a tissue-specific manner. Promoter is a DNA sequence generally described as the 5′-region of a gene, located proximal to the start codon. The transcription of an adjacent DNA segment is initiated at the promoter region. A repressible promoter's rate of 25 transcription decreases in response to a repressing agent. An inducible promoter's rate of transcription increases in response to an inducing agent. A constitutive promoter's rate of transcription is not specifically regulated, though it can vary under the Influence of general metabolic conditions.

The term “promoter” also includes the transcriptional regulatory sequences of a classical prokaryotic gene, in which case it may include a −35 box sequence and/or a −10 box transcriptional regulatory sequences.

Examples of promoters that can be used in the present invention are, but are not limited to cell-specific, tissue-specific, organospecific, constitutive, inducible, stress-inducible, pathogen-inducible, weak or strong promoters

The term “promoter” is also used to describe a synthetic or fusion molecule or derivative, which confers, activates or enhances expression of a nucleic acid molecule in a cell, tissue or organ.

Promoters may contain additional copies of one or more specific regulatory elements, to further enhance expression and/or to alter the spatial expression and/or temporal expression of a nucleic acid molecule to which it is operably connected. Such regulatory elements may be placed adjacent to a heterologous promoter sequence to drive expression of a nucleic acid molecule in response to e.g. copper, glucocorticoids, dexamethasone, tetracycline, gibberellin, cAMP, abscisic acid, auxin, wounding, ethylene, jasmonate or salicylic acid or to confer expression of a nucleic acid molecule to specific cells, tissues or organs such as meristems, leaves, roots, embryo, flowers, seeds or fruits.

In the context of the present invention, the promoter preferably is a plant-expressible promoter sequence. Promoters, however, that also function or solely function in non-plant cells such as bacteria, yeast cells, insect cells and animal cells are not excluded from the invention. By “plant-expressible” is meant that the promoter sequence, including any additional regulatory elements added thereto or contained therein, is at least capable of inducing, conferring, activating or enhancing expression in a plant cell, tissue or organ, preferably a monocotyledonous or dicotyledonous plant cell, tissue, or organ.

The terms “plant-operable” and “operable in a plant” when used herein, in respect of a promoter sequence, shall be taken to be equivalent to a plant-expressible promoter sequence.

In the present context, a “regulated promoter” or “regulatable promoter sequence” is a promoter that is capable of conferring expression on a structural gene in a particular cell, tissue, or organ or group of cells, tissues or organs of a plant, optionally under specific conditions, however does generally not confer expression throughout the plant under all conditions. Accordingly, a regulatable promoter sequence may be a promoter sequence that confers expression on a gene to which it is operably connected in a particular location within the plant or alternatively, throughout the plant under a specific set of conditions, such as following induction of gene expression by a chemical compound or other elicitor.

Preferably, the regulatable promoter used in the performance of the present invention confers expression in a specific location within the plant, either constitutively or following induction, however not in the whole plant under any circumstances. Included within the scope of such promoters are cell-specific promoter sequences, tissue-specific promoter sequences, organ-specific promoter sequences, cell cycle specific gene promoter sequences, inducible promoter sequences and constitutive promoter sequences that have been modified to confer expression in a particular part of the plant at any one time, such as by integration of said constitutive promoter within a transposable genetic element (Ac, Ds, Spm, En, or other transposon). Those skilled in the art will be aware that an “inducible promoter” is a promoter the transcriptional activity of which is increased or induced in response to a developmental, chemical, environmental, or physical stimulus. Similarly, the skilled craftsman will understand that a “constitutive promoter” is a promoter that Is transcriptionally active throughout most, but not necessarily all parts of an organism, preferably a plant, during most, but not necessarily all phases of its growth and development. Contrarily the term “ubiquitous promoter” is taken to indicate a promoter that is transcriptionally active throughout most, but not necessarily all parts of an organism, preferably a plant.

Generally by “weak promoter” Is intended a promoter that drives expression of a coding sequence at a low level. By “low level” is intended at levels of about 1/10,000 transcripts to about 1/100,000 transcripts, to about 1/500,0000 transcripts. Conversely, a “strong promoter” drives expression of a coding sequence at high level, or at about 1/10 transcripts to about 1/100 transcripts to about 1/1,000 transcripts.

The term “cell-specific” shall be taken to indicate that expression is predominantly in a particular cell or cell-type, preferably of plant origin, albeit not necessarily exclusively in said cell or cell-type.

Similarly, the term “tissue-specific” shall be taken to indicate that expression is predominantly in a particular tissue or tissue-type, preferably of plant origin, albeit not necessarily exclusively in said tissue or tissue-type.

Similarly, the term “organ-specific” shall be taken to indicate that expression is predominantly in a particular organ, preferably of plant origin, albeit not necessarily exclusively in said organ. “Root-specific” means that the promoter is expressed in the root only and not in other tissues of the plant.

By “root-preferred” it is intended that expression of the heterologous nucleotide sequence is most abundant root, but could also have low expression levels elsewhere in the plant. While some level of expression of the heterologous nucleotide sequence occurs in other plant tissue types, expression occurs most abundantly in the root including primary, lateral and adventitious roots.

The term “cell cycle specific” shall be taken to indicate that expression is predominantly cyclic and occurring in one or more, not necessarily consecutive phases of the cell cycle albeit not necessarily exclusively in cycling cells, preferably of plant origin.

Placing a nucleic acid molecule under the regulatory control of a promoter sequence, or in operable connection with a promoter sequence means positioning said nucleic acid molecule such that expression is controlled by the promoter sequence. A promoter is usually, but not necessarily, positioned upstream, or at the 5′-end, and within 2 kb of the start site of transcription, of the nucleic acid molecule which it regulates. In the construction of heterologous promoter/structural gene combinations it is generally preferred to position the promoter at a distance from the gene transcription start site that is approximately the same as the distance between that promoter and the gene it controls in its natural setting (i.e., the gene from which the promoter is derived). As is known in the art, some variation in this distance can be accommodated without loss of promoter function. Similarly, the preferred positioning of a regulatory sequence element with respect to a heterologous gene to be placed under its control is defined by the positioning of the element in its natural setting (i.e., the gene from which it is derived). Again, as is known in the art, some variation In this distance can also occur.

“Expression” means the production of a protein or nucleotide sequence in the cell itself or in a cell-free system. It includes transcription into an RNA product, post-transcriptional modification and/or translation to a protein product or polypeptide from a DNA encoding that product, as well as possible post-translational modifications.

“Operably linked” refers to a juxtaposition wherein the components so described are in a relationship permitting them to function in their intended manner. A control sequence “operably linked” to a coding sequence is ligated in such a way that expression of the coding sequence is achieved under conditions compatible with the control sequences. In case the control sequence is a promoter, it is obvious for a skilled person that double-stranded nucleic acid is preferably used.

The term “terminator” refers to a DNA sequence at the end of a transcriptional unit which signal termination of transcription. Terminators are 3′-non-translated DNA sequences containing a polyadenylation signal, which facilitates the addition of polyadenylate sequences to the 3′-end of a primary transcript. Terminators active in cells derived from viruses, yeasts, moulds, bacteria, insects, birds, mammals and plants are known and described in the literature. They may be isolated from bacteria, fungi, viruses, animals and/or plants.

Examples of terminators particularly suitable for use in the gene constructs of the present invention include the Agrobactecium tumefaciens nopaline synthase (NOS) gene terminator, the Agrobacteruium tumefaciens octopine synthase (OCS) gene terminator sequence, the Cauliflower mosaic virus (CaMV) ³⁵S gene terminator sequence, the Oryza sativa ADP-glucose pyrophosphorylase terminator sequence (t3′Bt2), the Zea mays zein gene terminator sequence, the rbcs-1A gene terminator, and the rbcs-3A gene terminator sequences, amongst others.

Those skilled in the art will be aware of additional promoter sequences and terminator sequences, which may be suitable for use in performing the invention. Such sequences may readily be used without any undue experimentation.

“Downregulation of expression” as used herein means lowering levels of gene expression and/or levels of active gene product and/or levels of gene product activity. Decreases in expression may be accomplished by e.g. the addition of coding sequences or parts thereof in a sense orientation (if resulting in co-suppression) or in an antisense orientation relative to a promoter sequence and furthermore by e.g. insertion mutagenesis (e.g. T-DNA insertion or transposon insertion) or by gene silencing strategies as described by e.g. Angell and Baulcombe 1998 (WO9836083), Lowe et al. 1989 (WO9853083), Lederer et al. 1999 (WO9915682) or Wang et al. 1999 (WO9953050). Genetic constructs aimed at silencing gene expression may have the nucleotide sequence of said gene (or one or more parts thereof) contained therein in a sense and/or antisense orientation relative to the promoter sequence. Another method to downregulate gene expression comprises the use of ribozymes, e.g. as described in Atkins et al. 1994 (WO9400012), Lenee et al. 1995 (WO9503404), Lutziger et al. 2000 (WO0000619), Prinsen et al. 1997 (WO9713865) and Scott et al. 1997 (WO9738116).

Means for introducing recombinant DNA into plant tissue or cells include, but are not limited to, transformation using CaCl₂ and variations thereof, in particular the method described previously (Hanahan 1983), direct DNA uptake into protoplasts (Krens et al. 1982; Paszkowski et al. 1984), PEG-mediated uptake to protoplasts (Armstrong et al. 1990) microparticle bombardment, electroporation (Fromm et al. 1985), microinjection of DNA (Crossway et al. 1986; Fromm et al. 1985), microparticle bombardment of tissue explants or cells (Christou et al. 1988), vacuum-infiltration of tissue with nucleic acid, or in the case of plants, T-DNA-mediated transfer from Agrobacterium to the plant tissue as described essentially (An et al. 1985; Dodds 1985; Herrera-Estrella et al. 1983a; Herrera-Estrella et al. 1983b). Methods for transformation of monocotyledonous plants are well known in the art and include Agrobacterium-mediated transformation (Cheng et al. 1997—WO9748814; Hansen 1998—WO9854961, Hiei et al. 1994—WO9400977; Hiei et al. 1998—WO9817813; Rikiishi et al. 1999—WO9904618; Saito et al. 1995—WO9506722), microprojectile bombardment (Adams et al. 1999—U.S. Pat. No. 5,969,213; Bowen et al. 1998—U.S. Pat. No. 5,736,369; Chang et al. 1994—WO9413822; Lundquist et al. 1999—U.S. Pat. Nos. 5,874,265/5,990,390; Vasil and Vasil 1995—U.S. Pat. No. 5,405,765; Walker et al. 1999—U.S. Pat. No. 5,955,362), DNA uptake (Eyal et al. 1993—WO9318168), microinjection of Agrobacterium cells (von Holt 1994—DE4309203) and sonication (Finer et al. 1997—U.S. Pat. No. 5,693,512).

For microparticle bombardment of cells, a microparticle is propelled into a cell to produce a transformed cell. Any suitable ballistic cell transformation methodology and apparatus can be used in performing the present invention. Exemplary apparatus and procedures are disclosed by Stomp et al. (U.S. Pat. No. 5,122,466) and Sanford and Wolf (U.S. Pat. No. 4,945,050). When using ballistic transformation procedures, the gene construct may incorporate a plasmid capable of replicating in the cell to be transformed.

Examples of microparticles suitable for use In such systems include 1 to 5 μm gold spheres. The DNA construct may be deposited on the microparticle by any suitable technique, such as by precipitation.

A whole plant may be regenerated from the transformed or transfected cell, in accordance with procedures well known in the art. Plant tissue capable of subsequent clonal propagation, whether by organogenesis or embryogenesis, may be transformed with a gene construct of the present invention and a whole plant regenerated therefrom. The particular tissue chosen will vary depending on the clonal propagation systems available for, and best suited to, the particular species being transformed. Exemplary tissue targets include leaf disks, pollen, embryos, cotyledons, hypocotyls, megagametophytes, callus tissue, existing meristematic tissue (e.g., apical meristem, axillary buds, and root meristems), and induced meristem tissue (e.g., cotyledon meristem and hypocotyl meristem).

With “T-DNA”, or transferred DNA, is meant that part of the transformation vector flanked by T-DNA borders which is, after activation of the Agrobacterium vir genes, nicked at the T-DNA borders and is transferred as a single stranded DNA to the nucleus of an eukaryotic cell.

When used herein, with “T-DNA borders”, “T-DNA border region”, or “border region” are meant either right T-DNA border (RB) or left T-DNA border (LB). Such a border comprises a core sequence flanked by a border inner region as part of the T-DNA flanking the border and/or a border outer region as part of the vector backbone flanking the border. The core sequences comprise 22 bp in case of octopine-type vectors and 25 bp in case of nopaline-type vectors. The core sequences in the right border region and left border region form imperfect repeats. Border core sequences are indispensable for recognition and processing by the Agrobacterium nicking complex consisting of at least VirD1 and VirD2. Core sequences flanking a T-DNA are sufficient to promote transfer of said T-DNA. However, efficiency of transformation using transformation vectors carrying said T-DNA solely flanked by said core sequences is low. Border inner and outer regions are known to modulate efficiency of T-DNA transfer (Wang et al. 1987). One element enhancing T-DNA transfer has been characterised and resides in the right border outer region and Is called overdrive (Peralta et al. 1986; van Haaren et al. 1987).

The vectors of the present Invention can be “T-DNA transformation vector” or “T-DNA vector”, which means any vector encompassing a T-DNA sequence flanked by a right and left T-DNA border consisting of at least the right and left border core sequences, respectively, and used for transformation of any eukaryotic cell.

With “T-DNA vector backbone sequence” or “T-DNA vector backbone sequences” is meant all DNA of a T-DNA containing vector that lies outside of the T-DNA borders and, more specifically, outside the nicking sites of the border core imperfect repeats.

Optimised T-DNA vectors can be constructed such that vector backbone integration in the genome of a eukaryotic cell is minimised or absent. With “optimised T-DNA vector” is meant a T-DNA vector designed either to decrease or abolish transfer of vector backbone sequences to the genome of a eukaryotic cell. Such T-DNA vectors are known to the one familiar with the art and include those described previously (Hanson et al. 1999), Stuiver et al. (1999—WO9901563).

With “binary transformation vector” is meant a T-DNA transformation vector comprising: a T-DNA region comprising at least one gene of interest and/or at least one selectable marker active in the eukaryotic cell to be transformed; and a vector backbone region comprising at least origins of replication active in E. coli and Agrobacterium and markers for selection in E. coli and Agrobacterium. Alternatively, replication of the binary transformation vector in Agrobacterium is dependent on the presence of a separate helper plasmid. The binary vector pGreen and the helper plasmid pSoup form an example of such a system as described in e.g. (Hellens et al 2000) or as available on the internet site http://www.pgreen.ac.uk.

The T-DNA borders of a binary transformation vector can be derived from octopine-type or nopaline-type Ti plasmids or from both. The T-DNA of a binary vector is only transferred to a eukaryotic cell in conjunction with a helper plasmid. Also known in the art are multiple binary vector Agrobacterium strains for efficient co-transformation of plants (Bidney and Scelonge 2000—WO0018939).

With “helper plasmid” is meant a plasmid that is stably maintained in Agrobacterium and is at least carrying the set of vir genes necessary for enabling transfer of the T-DNA. Said set of vir genes can be derived from either octopine-type or nopaline-type Ti plasmids or from both.

With “super-binary transformation vector” is meant a binary transformation vector additionally carrying in the vector backbone region a vfir region of the Ti plasmid pTiBo542 of the super-virulent A. tumefaciens strain A281 (Hiel et al. 1994—EP0604662, Hiei et al. 1995—EP0687730). Super-binary transformation vectors are used in conjunction with a helper plasmid.

With “co-integrate transformation vector” is meant a T-DNA vector at least comprising: a T-DNA region comprising at least one gene of interest and/or at least one selectable marker active in plants; and a vector backbone region comprising at least origins of replication active in Escherichia coli and Agrobacterium, and markers for selection in E. coli and Agrobacterium, and a set of vir genes necessary for enabling transfer of the T-DNA.

The T-DNA borders and said set of vir genes of a said T-DNA vector can be derived from either octopine-type or nopaline-type Ti plasmids or from both.

With “Ri-derived plant transformation vector” is meant a binary transformation vector in which the T-DNA borders are derived from a Ti plasmid and said binary transformation vector being used in conjunction with a ‘helper’ Ri-plasmid carrying the necessary set of virgenes.

As used herein, the term “selectable marker gene” or “selectable marker” or “marker for selection” includes any gene which confers a phenotype on a cell in which it is expressed to facilitate the identification and/or selection of cells which are transfected or transformed with a gene construct of the invention or a derivative thereof. Suitable selectable marker genes contemplated herein include the ampicillin resistance (Amp^{r}), tetracycline resistance gene (Tc^{r}), bacterial kanamycin resistance gene (Kan^{r}), phosphinothricin resistance gene, neomycin phosphotransferase gene (nptll), hygromycin resistance gene, β-glucuronidase (GUS) gene, chloramphenicol acetyltransferase (CAT) gene, green fluorescent protein (GFP) gene (Haseloff et al. 1997), and luciferase gene, amongst others. A selectable marker is a DNA segment that allows one to select for or against a molecule or a cell that contains it, often under particular conditions. These markers can encode an activity, such as, but not limited to, production of RNA, peptide, or protein, or can provide a binding site for RNA, peptides, proteins, inorganic and organic compounds or compositions and the like. Examples of Selectable markers include but are not limited to: (1) DNA segments that encode products which provide resistance against otherwise toxic compounds (e.g., antibiotics); (2) DNA segments that encode products which are otherwise lacking in the recipient cell (e.g., tRNA genes, auxotrophic markers); (3) DNA segments that encode products which suppress the activity of a gene product; (4) DNA segments that encode products which can be readily identified (e.g., phenotypic markers such as P-galactosidase, green fluorescent protein (GFP), and cell surface proteins); (5) DNA segments that bind products which are otherwise detrimental to cell survival and/or function; (6) DNA segments that otherwise inhibit the activity of any of the DNA segments described in Nos. 1-5 above (e.g., antisense oligonucleotides); (7) DNA segments that bind products that modify a substrate (e.g. restriction endonucleases); (8) DNA segments that can be used to isolate a desired molecule (e.g. specific protein binding sites); (9) DNA segments that encode a specific nucleotide sequence which can be otherwise non-functional (e.g., for PCR amplification of subpopulations of molecules); and/or (10) DNA segments, which when absent, directly or indirectly confer sensitivity to particular compounds.

With “agrolistics”, “agrolistic transformation” or “agrolistic transfer” is meant here a transformation method combining features of Agrobacterium-mediated transformation and of biolistic DNA delivery. As such, a T-DNA containing target plasmid is co-delivered with DNA/RNA enabling in planta production of VirD1 and VirD2 with or without VirE2 (Hansen & Chilton 1996; Hansen et al. 1997), (Hansen and Chilton 1997—WO9712046).

With “foreign DNA” is meant any DNA sequence that is introduced in the host's genome by recombinant techniques. Said foreign DNA includes e.g. a T-DNA sequence or a part thereof such as the T-DNA sequence comprising the selectable marker in an expressible format. Foreign DNA furthermore includes intervening DNA sequences as defined supra.

“Plant cell” comprises any cell derived from any plant and existing in culture as a single cell, a group of cells or a callus. A plant cell may also be any cell in a developing or mature plant in culture or growing in nature.

“Plant” or “Plants” comprise all plant species which belong to the superfamily Viridiplantae. The present invention is applicable to any plant, in particular a monocotyledonous plants and dicotyledonous plants including a fodder or forage legume, ornamental plant, food crop, tree, or shrub selected from the list comprising Acacia spp., Acer spp., Actinidia spp., Aesculus spp., Agathis australis, Albizia amara, Alsophila tricolor, Andropogon spp., Arachis spp, Areca catechu, Astelia fragrans, Astragalus cicer, Baikiaea plurijuga, Betula spp., Brassica spp., Bruguiera gymnorrhiza, Burkea africana, Butea frondosa, Cadaba farinosa, Calliandra spp, Camellia sinensis, Canna indica, Capsicum spp., Cassia spp., Centroema pubescens, Chaenomeles spp., Cinnamomum cassia, Coffea arabica, Colophospermum mopane, Coronillia varia, Cotoneaster serotina, Crataegus spp., Cucumis spp., Cupressus spp., Cyathea dealbata, Cydonia oblonga, Cryptomeria japonica, Cymbopogon spp., Cynthea dealbata, Cydonia oblonga, Dalbergia monetara, Davallia divaricata, Desmodium spp., Dicksonia squarosa, Diheteropogon amplectens, Dioclea spp, Dolichos spp., Dorycnium rectum, Echinochloa pyramidalis, Ehrartia spp., Eleusine coracana, Eragrestis spp., Erythrina spp., Eucalyptus spp., Euclea schimperi, Eulalia villosa, Fagopyrum spp., Feijoa sellowiana, Fragaria spp., Flemingia spp, Freycinetla banksai, Geranium thunbergii, Ginkgo biloba, Glycine javanica, Gliricidia spp, Gossypium hirsutum, Grevillea spp., Guibourtia coleosperma, Hedysa rum spp., Hemarthia altissima, Heteropogon contortus, Hordeum vulgare, Hyparrhenla rufa, Hypericum erectum, Hyperthelia dissoluta, Indigo incamata, Iris spp., Leptarrhena pyrolifolia, Lespediza spp., Lettuca spp., Leucaena leucocephala, Loudetia simplex, Lotonus bainesii, Lotus spp., Macrotyloma axillare, Malus spp., Manihot esculenta, Medicago sativa, Metasequoia glyptostroboides, Musa sapientum, Nicotianum spp., Onobrychis spp., Ornithopus spp., Oryza spp., Peltophorum africanum, Pennisetum spp., Persea gratissima, Petunia spp., Phaseolus spp., Phoenix canariensis, Phormium cookianum, Photinia spp., Picea glauca, Pinus spp., Pisum sativum, Podocarpus totara, Pogonarthria fleckii, Pogonarthria squarrosa, Populus spp., Prosopis cineraria, Pseudotsuga menziesli, Pterolobium stellatum, Pyrus communis, Quercus spp., Rhaphiolepsis umbellata, Rhopalostylis sapida, Rhus natalensis, Ribes grossularia, Ribes spp., Robinia pseudoacacia, Rosa spp., Rubus spp., Salix spp., Schyzachyrium sanguineum, Sciadopitys verticillata, Sequoia sempervirens, Sequoladendron giganteum, Sorghum bicolor, Spinacia spp., Sporobolus fimbriatus, Stiburus alopecuroides, Stylosanthos humilis, Tadehagi spp, Taxodium distichum, Themeda triandra, Trifolium spp., Triticum spp., Tsuga heterophylla, Vaccinium spp., Vicia spp. Vitis vinifera, Watsonia pyramidata, Zantedeschia aethiopica, Zea mays, amaranth, artichoke, asparagus, broccoli, brussel sprout, cabbage, canola, carrot, cauliflower, celery, collard greens, flax, kale, lentil, oilseed rape, okra, onion, potato, rice, soybean, straw, sugarbeet, sugar cane; sunflower, tomato, squash, and tea, amongst others, or the seeds of any plant specifically named above or a tissue, cell or organ culture of any of the above species The present invention is applicable to any plant, in particular a monocotyledonous plants and dicotyledonous plants including a fodder or forage legume, ornamental plant, food crop, tree, or shrub selected from the list comprising Acacia spp., Acer spp., Actinidia spp., Aesculus spp., Agathis australls, Albizia amara, Alsophila tricolor, Andropogon spp., Arachis spp, Areca catechu, Astelia fragrans, Astragalus cicer, Baikiaea plurijuga, Betula spp., Brassica spp., Bruguiera gymnorrhiza, Burkea africana, Butea frondosa, Cadaba farinosa, Calliandra spp, Camellia sinensis, Canna indica, Capsicum spp., Cassia spp., Centroema pubescens, Chaenomeles spp., Cinnamomum cassia, Coffea arabica, Colophospermum mopane, Coronillia varia, Cotoneaster serotina, Crataegus spp., Cucumis spp., Cupressus spp., Cyathea dealbata, Cydonia oblonga, Cryptomeria japonica, Cymbopogon spp., Cynthea dealbata, Cydonia oblonga, Dalbergia monetaria, Davallia divaricata, Desmodium spp., Dicksonia squarosa, Diheteropogon amplectens, Dioclea spp, Dolichos spp., Dorycnium rectum, Echinochloa pyramidalis, Ehrartia spp., Eleusine coracana, Eragrestis spp., Erythrina spp., Eucalyptus spp., Euclea schimperi, Eulalia villosa, Fagopyrum spp., Feijoa sellowiana, Fragaria spp., Flemingia spp, Freycinetia banksli, Geranium thunbergii, Ginkgo biloba, Glycine javanica, Gliricidia spp, Gossypium hirsutum, Grevillea spp., Gulbourtia coleosperna, Hedysarum spp., Hemarthia altissima, Heteropogon contortus, Hordeum vulgare, Hyparrhenia rufa, Hypericum erectum, Hyperthelia dissoluta, Indigo incamata, Iris spp., Leptarrhena pyrolifolia, Lespediza spp., Lettuca spp., Leucaena leucocephala, Loudetia simplex, Lotonus bainesi, Lotus spp., Macrotyloma axillare, Malus spp., Manihot esculenta, Medicago sativa, Metasequoia glyptostroboides, Musa sapientum, Nicotianum spp., Onobrychis spp., Omithopus spp., Oryza spp., Peltophorum africanum, Pennisetum spp., Persea gratissima, Petunia spp., Phaseolus spp., Phoenix canariensis, Phormium cookianum, Photinla spp., Picea glauca, Pinus spp., Pisum sativum, Podocarpus totara, Pogonarthria fleckii, Pogonarthria squarrosa, Populus spp., Prosopis cineraria, Pseudotsuga merziesai, Pterolobium stellatum, Pyrus communis, Quercus spp., Rhaphiolepsis umbellata, Rhopalostylis sapida, Rhus natalensis, Ribes grossularia, Ribes spp., Robinia pseudoacacia, Rosa spp., Rubus spp., Salix spp., Schyzachyrium sanguineum, Sciadopitys verticillata, Sequoia sempervirens, Sequoiadendron giganteum, Sorghum bicolor, Spinacia spp., Sporobolus fimbriatus, Stiburus alopecuroides, Stylosanthos humilis, Tadehagi spp, Taxodium distichum, Themeda triandra, Trifolium spp., Triticum spp., Tsuga heterophylla, Vaccinium spp., Vicia spp. Vitis vinifera, Watsonia pyramidata, Zantedeschia aethiopica, Zea mays, amaranth, artichoke, asparagus, broccoli, brussel sprout, cabbage, canola, carrot, cauliflower, celery, collard greens, flax, kale, lentil, oilseed rape, okra, onion, potato, rice, soybean, straw, sugarbeet, sugar cane, sunflower, tomato, squash, and tea, amongst others, or the seeds of any plant specifically named above or a tissue, cell or organ culture of any of the above species

“Cereal” comprises crop plants with edible grain for example plants belonging to the grass family that is cultivated for its nutritious grains such as oats, barley, rye, wheat, rice, and corn etc.

### DESCRIPTION OF FIGURES

FIG. 1

Sequences attB1 and attB2 of the attB recombination site that are flanking a gene in the final expression vector after cloning from an entry clone into a destination vector via the Gateway™ LR clonase reaction.

FIG. 2

A spectinomycin-Streptomycin-(Sm/SpR) resistant destination vector containing a promoter, the Gateway™-cassette comprising the site-specifc recombination sequences attR1 and attR2, the chloramphenicol resistance gene (CamR) and the ccdb suicide gene, and a terminator

FIG. 3

The pUC18-vector containing the shortened (chloramphenicol resistance gene was removed) Gateway™-cassette and a Matrix associated region (MARs)-sequence.

FIG. 4

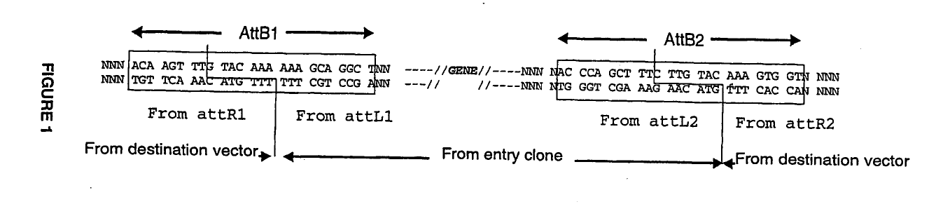

A. Schematic representation of a destination vector, comprising two recombination sites (RS) in opposite directions, separated by a spacer sequence and controlled by the same promoter and 3′untranslated region (3′), for the single step cloning of a co-suppression construct.

B. More detailed representation of a destination vector as in A for the single step cloning of a co-suppression construct. A spectinomycin-Streptomycin-resistant destination vector containing a promoter, followed by a first Gateway™-cassette in the attR2-attR1 direction, the MARs-sequence and then the second Gateway-cassette in the attR1-attR2 direction, and a terminator at the end.

FIG. 5

Donor vector pDONR201 (Life Technologies, Paisley, UK), a kanamycin-resistant vector which comprises of the Gateway™-cassette containing the attP1 and attP2 recombination sequences; chloramphenicol resistance gene and lethal ccdB-gene, flanked by the sequences of the attP-site.

FIG. 6

Schematic representation of a Gateway entry vector: the kanamycin-resistant (KMr) vector contains a gene flanked by attL1 and attL2-sequences of the attL recombination site.

FIG. 7

Restriction pattern of 8 candidates of the co-suppression construct with the SH2B gene after performing the double LR reaction. The restriction digest was loaded on an 1% agarosegel (Life Technologies, Paisley, UK)

Lane 1 is the marker (Smartladder, Eurogentec, Seraing, Belgium). Lanes 2 up to 9 are clones 1 up to 8. The sizes of the Smartladder is indicated in the left panel.

FIG. 8

Schematic representation of the co-suppression (expression) vector after the double Gateway™-reaction. The spectinomycin-streptomycin-resistant vector contains the following features: a promoter, the attB2 sequence, gene X in anti-clockwise orientation, attB1 sequence, MARs, attB1 sequence, gene X in clockwise orientation, attB2 and terminator.

FIG. 9

Schematic representation of a destination vector, comprising two recombination sites (RS) in opposite directions and each controlled by a separate promoter and optionally a 3′ untranslated region (3′) for the single step cloning of a gene combination construct. Alternatively, the two recombination sites (RS) are under the control of one bidirectional promoter which exerts a dual promoter function in opposite directions.

FIG. 10

Schematic representation of a destination vector, comprising two adjacent recombination sites in opposite direction (RS), each followed by a coding sequence (CDS) and optionally by a terminator (3′) UTR for the single step cloning of a promoter combination construct. In a first example the two recombination sites (RS) are separated by a spacer so that the two promoters function each separately. Alternatively, the two promoter elements can be cloned as such that they are joined in one bidirectional promoter which exerts a dual promoter function in opposite directions.

FIG. 11

Schematic representation of a destination vector, comprising two recombination sites in opposite directions (RS) each of which is (optionally) followed by a 3′ untranslated region for the single-step cloning of a promoter-gene combination vector.

FIG. 12

Schematic representation of a destination vector, comprising two recombination sites in opposite directions (RS) separated by a coding region (CDS) for the single-step cloning of a gene silencing vector.

FIG. 13

Schematic representation of a destination vector, comprising two recombination sites (RS) in opposite directions, separated by a ribosome binding site (RBS) and controlled by the same promoter and (optionally) 3′UTR, for the single step cloning of a polycistronic RNA construct.

FIG. 14

Schematic representation of a destination vector, comprising two recombination sites (RS) in opposite directions, for the single step cloning of two DNA segments (each comprising two recombination sites) that again allow a second round or multiple rounds of such single step cloning of two DNA segments. This destination vector is typically used for gene stacking.

FIG. 15

Schematic representation of a destination vector for construction of a bidirectional promoter, comprising two recombination sites (RS) in opposite directions, separated by a spacer sequence (grey box) optionally containing transcription initiation or transcription control elements, or containing a minimal promoter. Alternatively, there is no spacer sequence and two promoter elements are fused in opposite directions.

FIG. 16

Schematic representation of a destination vector for the construction of a vector carrying multiple expression cassettes, comprising two recombination sites (RS), in opposite directions. These expression cassettes can be generated by the combigate methodology (European patent application EP 02075373.7).

FIG. 17

Schematic representation of destination vectors for the construction of one or more fusion proteins, comprising two recombination sites (RS) in opposite directions.

In a first example, the RS sites are used to clone the promoter and the gene while the second part of the fusion protein is already in the vector. In this illustration the RS sites are opposite, but after cloning the promoter and the gene must be in the same orientation. As described earlier this can be achieved by cloning the promoter in opposite direction in the entry vector.

In a second example, two fusion proteins are constructed in a single-step recombination reaction, by cloning the two DNA segments in the RS sites that are flanked by a promoter and the second portion of the fusion protein.

In a third example, the first and the second portion of the fusion protein are cloned in a single-step recombination reaction. Optionally, the fusion protein can be cloned immediately under the control of a promoter that Is already present in the vector. Although the RS sites are in opposite direction in the destination vector, the two portions of the fusion protein must be cloned in the same orientation. (As described earlier, this can be achieved by choosing the right orientation in the entry vector).

The fusion proteins can be made based on the fusion of a DNA insert of interest (e.g. a gene) and the second portion of the fusion protein (grey box). This second part can be chosen from a wide variety of DNA fragments e.g. encoding an epitope-tag, a reporter gene, a functional domain etc. This portion of the fusion protein can be fused C-terminal with respect to the cloned DNA insert, b [symbol] lternatively, this portion of the fusion protein can be fused N-terminal of the cloned DNA insert (see FIG. 18).

FIG. 18

In analogy of FIG. 17 the second portion of the fusion protein (grey box) is coupled N-terminal of the cloned DNA insert.

### EXAMPLES

The following non-limiting examples describe the construction of destination vectors for the single-step uptake of multiple DNA segments and the use of those vectors or parts thereof. Unless stated otherwise in the examples, all recombinant DNA techniques are carried out according to standard protocols as described in Sambrook et al (1989, Molecular cloning: A laboratory manual, cold spring harbor laboratory press, NY)

### Example 1

### Construction of a Co-Suppression Destination Vector for Plants

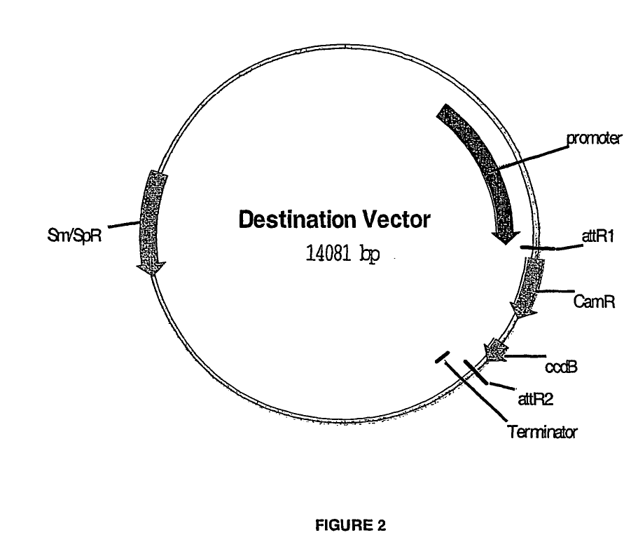

A plant expression vector (FIG. 2) was prepared by putting the commercially available linear Gateway™-cassette (Life Technologies, Paisley, UK) between a GOS2-promotor and the t-zein terminator. Next to the key features of this general destination vector shown in FIG. 2, the vector also contains other features, which are important for plant expression (e.g. T-DNA borders).

For preparing the destination vector for co-suppression of a gene, the above-described vector was used to start the cloning. A Stul-restriction site was created between the promoter and the Gateway™-cassette, to be able to enter a second Gateway-cassette and 315 base pairs of the MARs-sequence (Nicotina tabacum matrix associated region). This site was created by a series of PCR-reactions. First the vector was cut with PvulI and MluI, which gives three bands with following sizes: 12 kb, 1 kb and 0.5 kb. The 12 kb fragment was kept to put in the final PCR again. The 1 kb fragments was used as a template for two PCR-reactions. The first PCR, using forward primer 5′CTGGCTTGTTTAGATACAGTAGTC3′ (SEQ ID NO 1) and reverse primer which contains the StuI-mutation 5′CTTGTGATAGGCCTAGTTGATT3′ (SEQ ID NO 2) gave an amplification band of 615 base pairs. The second PCR, using a forward primer which is the complementary of the reverse primer of the first PCR and a reverse primer 5′ATATCAGCTGTAAAGCCTGGG3′ (SEQ. ID NO 3) containing an MluI-site gave an amplification of 166 base pairs. The bands obtained in these two PCR-reactions were then used as template for the third PCR. The primers used for this PCR were the forward primer of the first PCR and the reverse primer of the second PCR. This third PCR gave an amplification of 759 base pairs. After cutting this PCR-fragment with MluI, it was ligated into the 12 kb restriction fragment as mentioned above. The reverse primer of the second PCR was chosen in that way that the chloramphenicol-resistance-gene was almost totally removed (leaving 13 base pairs) from the Gateway™-cassette to make the final destination vector smaller. This shortened Gateway cassette is further referred to as RS (Recombination Site) Now a blunt-cutting StuI-site is available between the promoter and the RS-cassette.

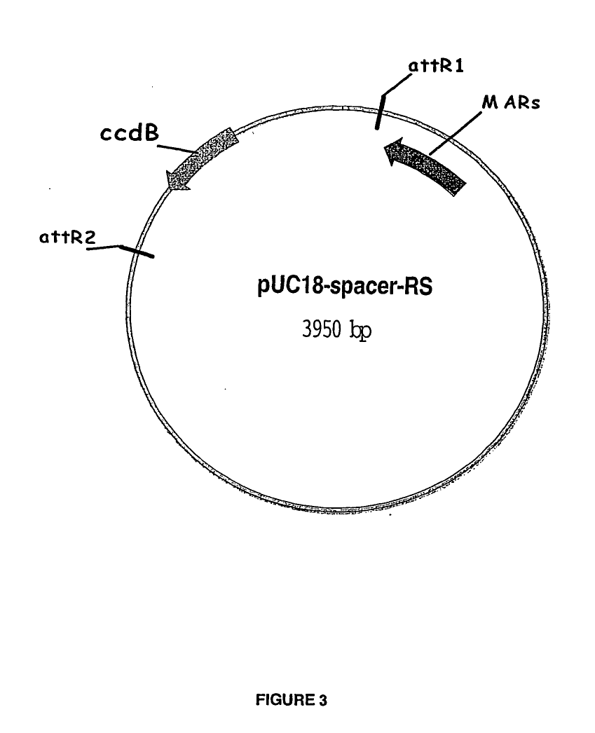

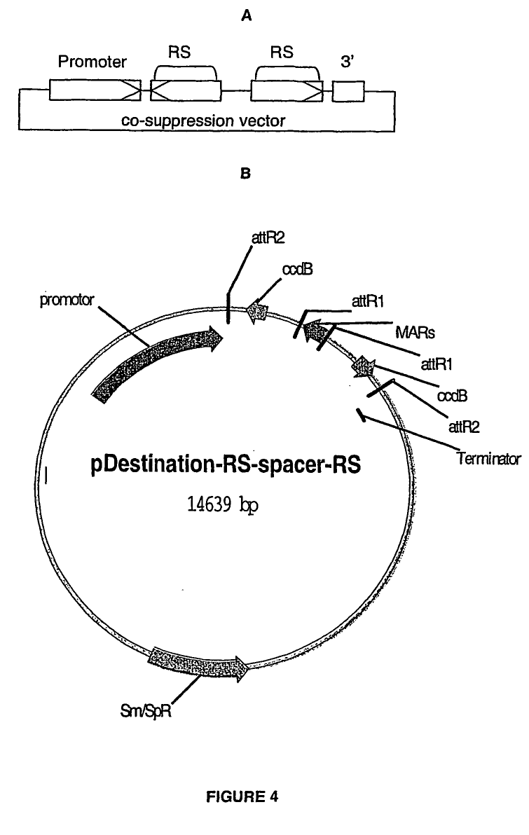

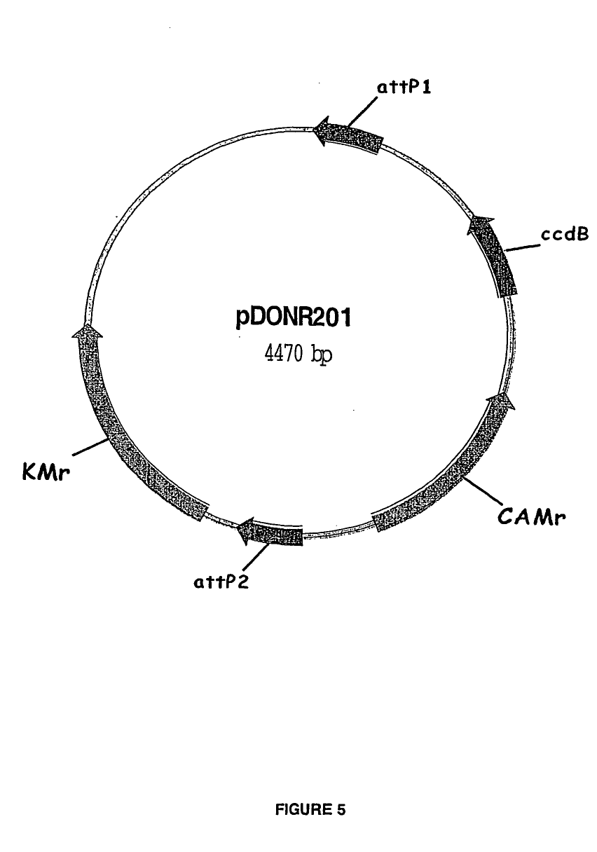

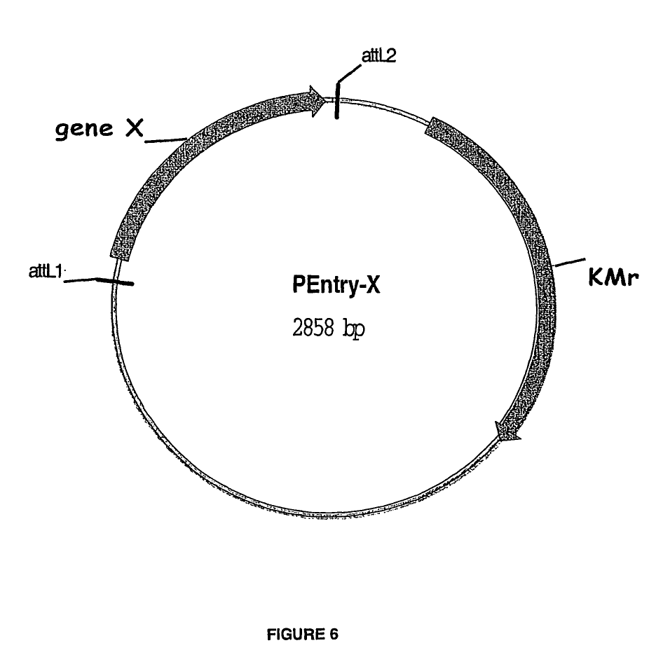

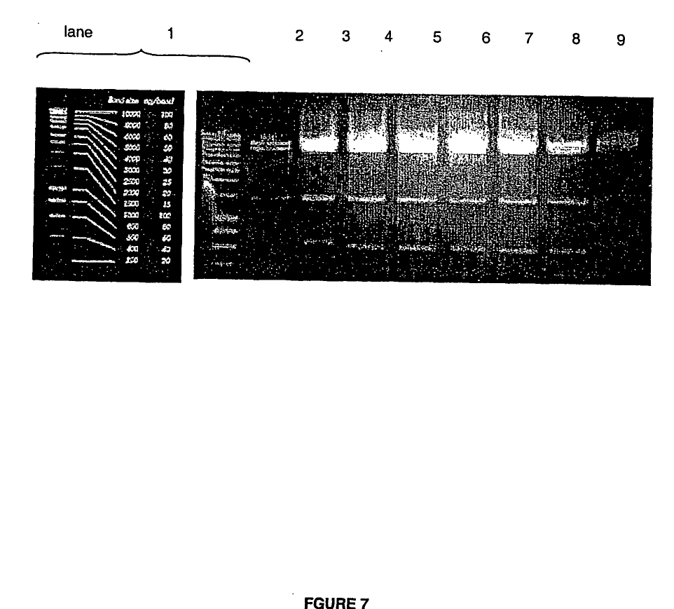

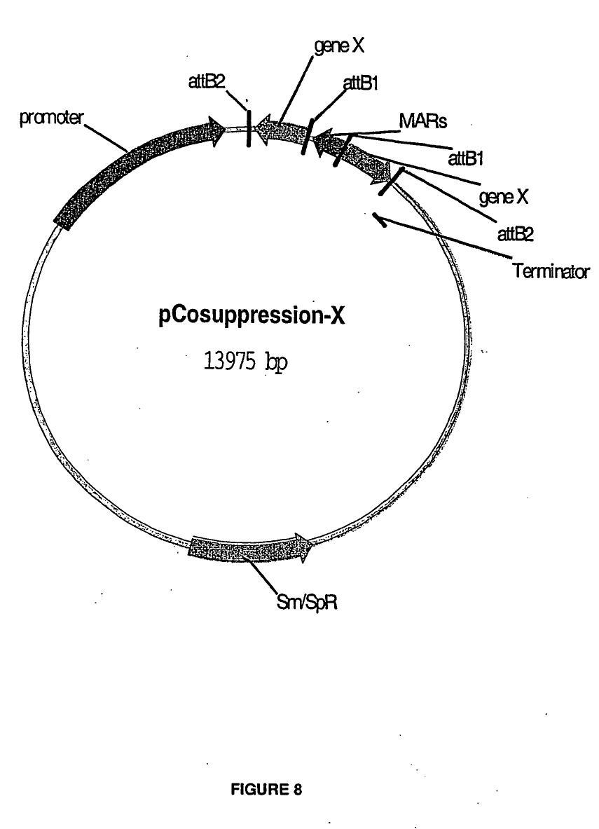

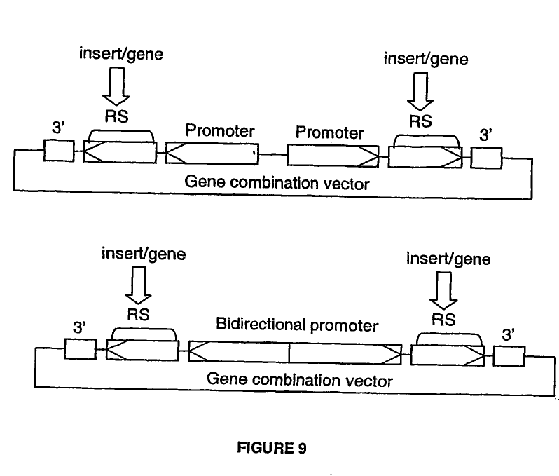

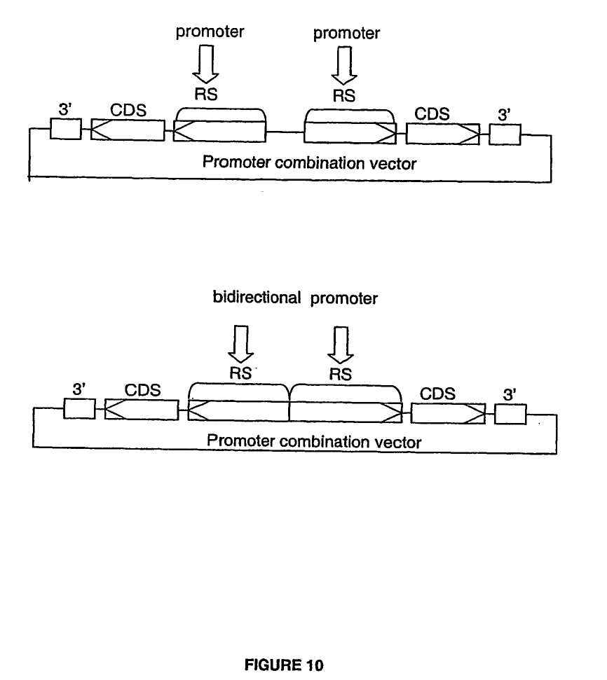

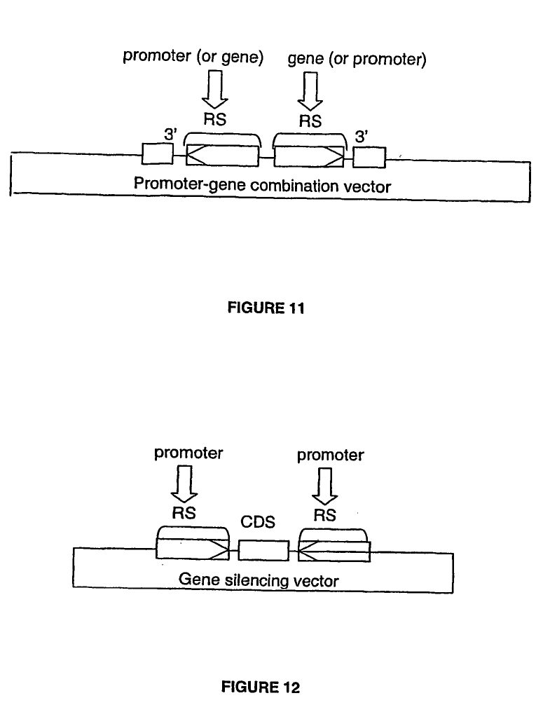

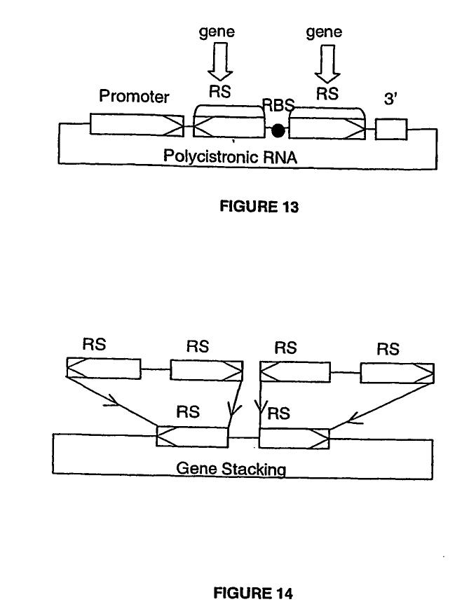

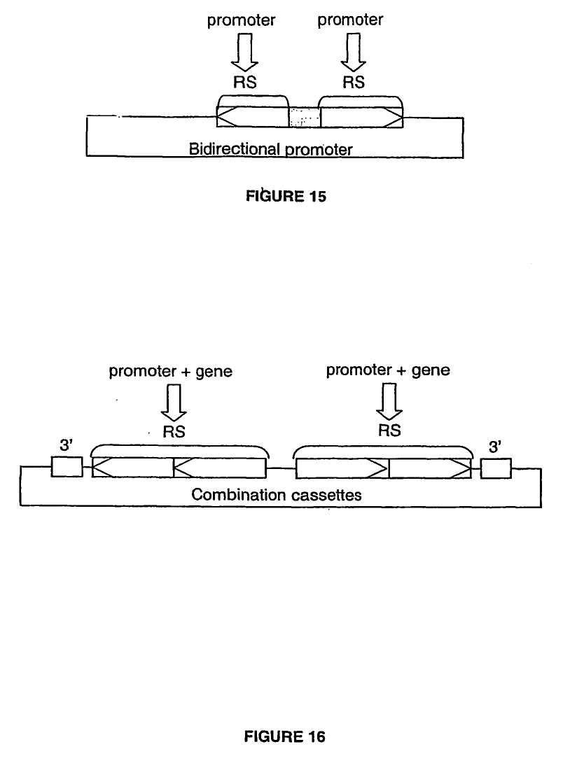

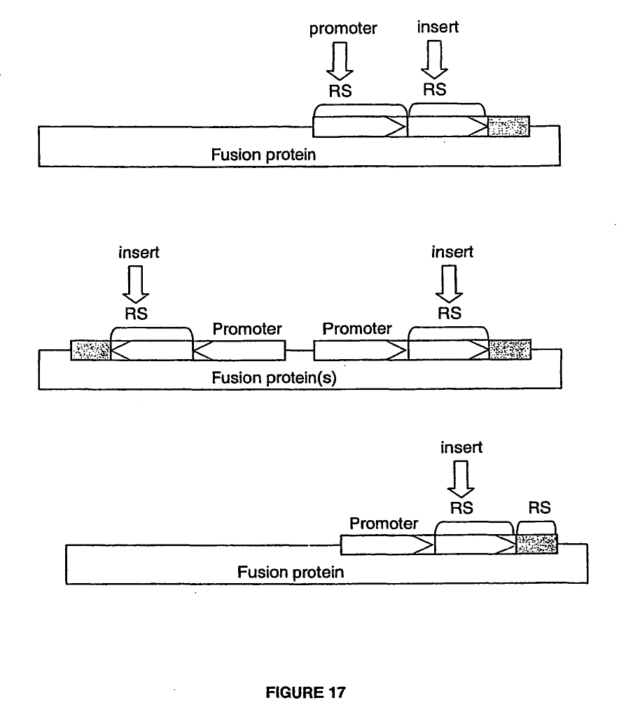

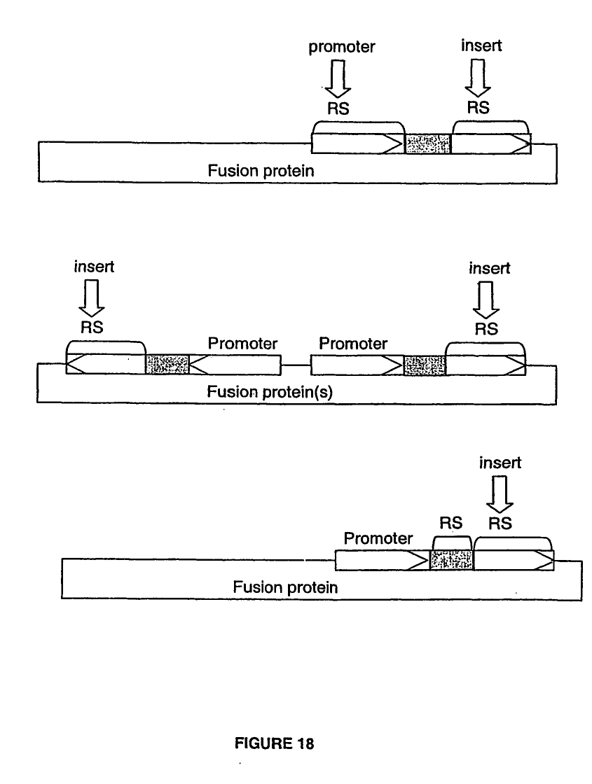

In a pUC18-vector, shown in FIG. 3, the linear commercial Gateway™-cassette was cloned in front of the MARs-sequence (Genbank accession number NU67919, amplified with the primers (forward primer 5′-GTTGTCMTATCCTGGAAATTTTGC-3′ (SEQ ID NO 4) and the reverse primer 5′-CTGCCATTCTTTAGAGGGGATGCTTG-3′ (SEQ ID NO 5), and also here the chloramphenicol-resistance-gene was deleted (total deletion) from the cassette, using restriction sites Mlul and NotI. This resulted in the vector pUC-18-spacer-RS (Recombination Site). The MARs-RS cassette was cut from this vector by HindIII-Ecl13611, followed by a Pfu-fill in-reaction and then ligated into the CIP-treated StuI-site in such a way that the following features were present in the final vector: first a promoter, followed by a first RS-cassette in the attR2-attR1 direction, the MARs-sequence and then the second RS-cassette in the attR1-attR2 direction, and a terminator at the end. This final co-suppression destination vector referred to as pDestination-RS-spacer-RS (FIG. 4) was used for the double LR-reaction (see example 3).

### Example 2

### Preparation of the Entry Vector

An entry vector consists of the gene of interest, flanked with attL1 and attL2-sites. The gene is picked up by a PCR-reaction where the forward primer has an addition of 5′ terminal attB1-sequence (5′ GGGGACMGTTTGTACAAAAAAGCAGGCTNN-template specific sequence-3′) (SEQ ID NO 6) and to the reverse primer the 5′ terminal attB2-site (5′GGGGACCACMGTACMGAAAGCTGGGTN-template specific sequence-3′) (SEQ ID NO 7) was added. The amplification product is then the gene of interest flanked by attB-sites. The combination of this PCR product with the donor vector pDONR201 (containing attP-sites) (Life Technologies, Paisley, UK) (FIG. 5) and the BP Clonase enzyme (Ufe Technologies, Paisley, UK) results in an entry vector of the PCR product, referred to as pEntry-X (FIG. 6). The BP Clonase enzyme (Life Technologies, Paisley, UK) recombines the attP-sites of the donor vector with the attB-sites of the PCR product. This is a directional cloning since attB1 reacts only with attP1, and attB2 reacts only with attP1.

### Example 3

### The Double LR Site-Specific Recombination Reaction for Generation of a Plant Co-Suppression Vector

The inventors constructed a vector intended for high-throughput cloning of co-suppression constructs. In a single reaction tube, using one single insert DNA sequence, the insert is cloned twice but in opposite directions in the destination vector. The destination vector has the following basic layout (FIG. 4) (1) a promoter driving the expression of downstream sequences (2) a first and a second (shortened) RS recombination cassettes, oriented opposite towards each other and spaced by a 315 bp spacer region (MAR sequence). The RS cassettes preferentially (but not necessarily) contain selectable or screenable markers, which allows the elimination of transformed cells, which do not contain the double insert and (3) an optional 3′ UTR

For performing the LR-reaction, an entry vector (which can be circular or linear) with the gene of interest and a destination vector are needed.

As destination vector, plant expression vector with the double RS-cassette, as described in example 1 is used.

For the entry vector the strategy of example 2 was followed. In order to compare three different experimental setups, three entry vectors were generated each containing a different gene: SH2A, SH2B and Ras p21.

Via the LR-reaction, using the entry vector (with gene of interest flanked by attL-sequences) and the destination vector (with attR-sequences) together with the LR clonase enzyme (Life Technologies, Paisley, UK) a recombination of the attL- and attR-sequences takes place, resulting in a final expression vector containing the gene of interest flanked by attB-sequences. Again the orientation of the gene is maintained since attL1 reacts only with attR1, and attL2 reacts only with attR2.

The destination vector consists of two RS-cassettes, so a double recombination has to take place in one reaction tube in the same reaction mix. The double LR reaction is done in a total volume of 5 μl, consisting of 1 μl entry vector (prepared using CONCERT Rapid Plasmid Mini Prep Purification System, ±100 ng/μl Life technologies, Paisley, UK), 1 μl destination vector (75 ng/μl), 1 μl LR reaction buffer (Life technologies, Paisley, UK), 1 μl LR clonase enzyme (Life technologies, Paisley, UK) and 1 μl TE buffer (Ufe technologies, Paisley, UK). The mixture was incubated at 25° C. overnight. After incubation, the 5 μl were transformed into 50 μl Library efficiency DH5α-cells (Life technologies, Paisley, UK). The cell-reaction mixture was incubated on ice for 1 min, then heat-shocked at 42° C. for 45 seconds and again put on ice for 2 minutes. Then 950 μl SOC (2% bactotryptone, 0.5% bacto yeast extract, 10 mM NaCL, 2.5 mM KCl, 10 mM MgCl₂, 10 mM MgSO₄, 20 mM glucose was added. After shaking incubating at 37° C. for one hour, the transformation mixture was plated on LuriaBroth-agar plates (ICN Biomedicals, Inc. Ohio, USA), containing spectinomycine and streptomycine (Duchefa Biochemie by, Haarlem, Nederland), both in a final concentration of 25 μg/ml. The plates were incubated at 37° C. for one night.

### Example 4

### Evaluation of the Transformants and of the Cloning Efficiency

The double LR reaction was tested three times with three different genes, namely SH2A, SH2B and Ras p21. An average of 50 colonies per reaction mixture was obtained. 8 colonies of each construct were inoculated in 2 ml liquid culture LuriaBroth (ICN Biomedicals, Inc. Ohio, USA), containing spectinomycine and streptomycine (Duchefa Biochemie by, Haarlem, Nederland), both in a final concentration of 25 μg/ml and Incubated at 37° C. overnight. DNA minipreps (CONCERT Rapid Plasmid Mini Prep Purification System, Life technologies, Paisley, UK), were made and the DNA was checked with restriction digest. For this digest, a restriction site was picked that occurred in the gene, and not in the middle of the sequence, to be able to check not only for the presence of the gene, but also for the orientation. For the SH2A gene, this restriction enzyme was PvuII, for SH2B it was BanII, while for Ras p21 SacIl was used. For all three constructs, all eight candidates showed the expected pattern. In FIG. 7, the restriction digest for the co-suppression vector with the SH2B gene is shown. If the gene Is inserted twice and in the opposite orientations, bands of 6.8 kb, 5.2 kb, 1.4 kb and 0.6 kb are expected, from which the band of 0.6 kb proofs the presence of the genes In the wanted orientations. These bands are present in all 8 candidates, indicating that all the 8 clones have the intended genes in the intended orientation. If the gene would be present twice in the clockwise orientation, the expected bands would be 6.8 kb, 5.2 kb, 1.0 kb and 1.0 kb. If the gene would be present twice in the anti-clockwise orientation, the expected bands would be 6.4 kb, 5.2 kb, 1.4 kb and 1.0 kb. These would also be the bands if the genes where in opposite directions, but where the first orientation after the promoter would be clockwise instead of anti-clockwise.

The LR recombination reaction with double recombination sites in the destination vector, using the reaction conditions as described in example 3, yielded half of the number of transformants compared to a LR recombination with a single recombination site in the destination vector, using the same reaction condition, at the level of number of CFU upon plating out. Per construct however, all tested candidate clones were having the inserts in the desired orientation. This overall efficiency allows high-throughput cloning.

From these results the inventors could conclude that a gene can be inserted twice in the same construct doing only one reaction. FIG. 8 shows the expression vector pCosuppression-X, which is the result of a double LR recombination-reaction of a gene X from the pEntry-X with a cosuppression destination vector pDestination-RS-spacer-RS.

### Example 5

### Construction of a Gene Combination Vector

The technology of the present invention is not restricted to the construction of co-suppression constructs. It is also used for the high-throughput construction of different gene combination constructs, given that the two RS cassettes are oriented in an opposite orientation towards each other (to prevent that the internal region between the RS cassettes is recombined out). The destination vector for a gene combination constructs has the following basic lay-out (FIG. 9): (1) two promoters, in opposite orientation (2) two recombination site (for example a (shortened) RS recombination cassette) in opposite orientation and (3) two 3′ UTR. This construct allows the insertion of two DNA segments in and placed between each pair of promoter and an optional 3′ UTR. The single step recombination reaction is performed as in example 3, except that instead of using one entry vector, also two entry vectors each carrying a different gene can be used. This vector is suitable for the simultaneous expression of two genes, controlled by the same or by different promoters, dependent on the choice of promoters in the destination vector.

### Example 6

### Construction of a Promoter Combination Vector

Using the method of the present Invention and in analogy with example 5, also a vector is conceived that allows swapping of promoters, instead of swapping coding sequences. This construct has the following basic layout (FIG. 10): (1) two recombination sites (for example a (shortened) RS cassette) in opposite direction each followed by (2) a coding sequence, each followed by (3) an optional 3′UTR. This destination vector is used for the high throughput cloning of two promoters. In this destination vector the coding sequences are the same or different and the cloned promoters are also the same or different. This construct is particularly useful to have two expression control elements for a particular coding sequence in one vector.

### Example 7

### Construction of a Promoter-Gene Combination Vectors

Using the method of the present invention and in analogy with example 5, also a vector is conceived that allows the simultaneous cloning of a promoter and a gene. This construct has the following layout (FIG. 11): (1) two adjacent recombination sites (for example a (shortened) RS cassettes) that are in opposite orientation, separated by an intervening sequences which does not disturb the control of the promoter that is cloned in the first recombination site on the coding sequence, which is cloned in the second recombination site and (3) an optional 3′ untranslated region, flanking each recombination site or flanking only one of them. For the construction of this type of expression vector, the orientation of the inserted DNA segments is very important. The recombination site in the destination vector wherein the promoter is inserted is in opposite direction with regard to the recombination site for the coding sequence. Therefore, the entry vector used for the promoter is designed in a way that after recombination the promoter is in the same direction as the coding sequence. This is done by cloning the promoter in its entry clone in opposite orientation compared to the orientation of the coding sequence in its entry clone. After the single step recombination of these two entry vectors with the destination vector as in FIG. 11, 2 out of 4 resulting products have the correct orientation of the promoter, followed by the coding sequence and followed by an 3′ untranslated region.

### Example 8

### Construction of a Gene-Silencing Vector with Convergent Promoters

In analogy to the above mentioned co-suppression vector, wherein the two recombination sites are separated with a spacer sequence and both under the control of the same promoter, a destination vector is constructed with two convergent promoters having the following structure (FIG. 12): (1) two recombination sites (for example (shortened) RS cassettes) in opposite orientations, separated by (2) a coding sequence. In this type of vector two promoters are cloned in opposite directions in the opposite recombination sites, and the promoters can each transcribe the coding sequence.

### Example 9

### Construction of a Vector with a Polycistronic RNA

Also in analogy to the above mentioned co-suppression vector, wherein the two recombination sites are separated with a spacer sequence and both under the control of the same promoter, a vector is constructed with a polycistronic RNA, having the following structure (FIG. 13) (1) a promoter followed by (2) two recombination sites (for example (shortened) RS cassettes) that are separated by (2) a ribosome bindings site. In this type of vector two genes are cloned in the same orientation by preparing one of those genes in the entry clone in the opposite orientation compared to the orientation of the other gene in the entry clone. When these two entry clones are recombined with the vector as in FIG. 13, the two genes will be in the same direction because the recombination sites in the destination vector are in opposite directions.

### REFERENCES

An G., Watson B. D., Stachel S., Gordon M. P., & Nester E. W. (1985) New cloning vehicles for transformation of higher plants. EMBO J. 4, 277-284.

Armstrong C. L, Petersen W. P., Buchholz W. G., Bowen B. A., & Sulc S. L. (1990) Factors affecting PEG-mediated stable transformation of maize protoplasts. Plant Cell Reports 9, 335-339.

Christou P., McCabe D. E., & Swain W. P. (1988) Stable transformation of soybean callus by DNA-coated gold particles. Plant Physiol. 87, 671-674.

Crossway A., Oakes J. V., Irvine J. M., Ward B., Knauf V. C., & Shewmaker C. K. (1986) Integration of foreign DNA following microinjection of tobacco mesophyll protoplasts. Mol. Gen. Genet. 202, 179-185.

Dodds J. H. (1985) Plant genetic engineering. Cambridge University Press.

Fromm M., Taylor L. P., & Walbot V. (1985) Expression of genes transferred into monocot and dicot plant cells by electroporation. Proc. Natl. Acad. Sci. U.S.A 82, 5824-5828.

Hanahan D. (1983) Studies on transformation of Escherichia coli with plasmids. J. Mol. Biol 166, 557-580.

Hansen G. & Chilton M. D. (1996) “Agrolistic” transformation of plant cells: integration of T-strands generated in planta. Proc. Natl. Acad. Sci. U.S.A 93, 14978-14983.

Hansen G., Shillito R. D., & Chilton M. D. (1997) T-strand integration in maize protoplasts after codelivery of a T-DNA substrate and virulence genes. Proc. Natl. Acad. Sci. U.S.A 94, 11726-11730.

Hanson B., Engler D., Moy Y., Newman B., Ralston E., & Gutterson N. (1999) A simple method to enrich an Agrobacterium-transformed population for plants containing only T-DNA sequences. Plant J. 19, 727-734.

Haseloff J., Siemering K. R., Prasher D. C., & Hodge S. (1997) Removal of a cryptic intron and subcellular localization of green fluorescent protein are required to mark transgenic Arabidopsis plants brightly. Proc. Natl. Acad. Sci. U.S.A 94, 2122-2127.

Hellens R. P., Edwards E. A., Leyland N. R., Bean S., & Mullineaux P. M. (2000) pGreen: a versatile and flexible binary Ti vector for Agrobacterium-mediated plant transformation. Plant Mol. Biol. 42, 819-832.

Herrera-Estrella L., De Block M., Messens E. H. J. P., Van Montagu M., & Schell J. (1983a) Chimeric genes as dominant selectable markers in plant cells. EMBO J. 2, 987-995.

Herrera-Estrella L., Depicker A., Van Montagu M., & Schell J. (1983b) Expression of cnimaeric genes transferred into plant cells using a Ti-plasmid-derived vector. Nature 303, 209-213.

Krens F. A., Molendijk L., Wullems G. J., & Schilperoort R. A. (1982) In vitro transformation of plant protoplasts with Ti-plasmid DNA. Nature 296, 72-74.

Paszkowski J., Shillito R. D., Saul M., Mandak V., & Hohn T. H. B. P. I. (1984) Direct gene transfer to plants. EMBO J. 3, 2717-2722.

Peralta E. G., Hellmiss R., & Ream W. (1986) Overdrive, a T-DNA transmission enhancer on the A. tumefaciens tumour-inducing plasmid. EMBO J. 5, 1137-1142.

Sugita K., Kasahara T., Matsunaga E., & Ebinuma H. (2000) Technical advance: A transformation vector for the production of marker-free transgenic plants containing a single copy transgene at high frequency [In Process Citation]. Plant J. 22, 461-469.

van Haaren M. J., Sedee N. J., Schilperoort R. A., & Hooykaas P. J. (1987) Overdrive is a T-region transfer enhancer which stimulates T-strand production in Agrobacterium tumefaciens. Nucleic Acids Res. 15, 8983-8997.

Wang K., Genetello C., Van Montagu M., & Zambryski P. C. (1987) Sequence context of the T-DNA border repeat element determines Its relative activity during T-DNA transfer to plant cells. Mol. Gen. Genet. 210, 338-346.

1. A DNA molecule for the single-step cloning of two DNA segments, said molecule comprising two first site-specific recombination sites which are
(a) each comprising the same pair of recombination sequences,
(b) each functional for uptake or exchange of a DNA segment,
(c) each reactive to the same second site-specific recombination site, and,
(d) targeted by the same recombinase mix,
and wherein the recombination sequences from each of the first recombination sites can recombine with the recombination sequences of the second recombination site, without interference of the recombination sequences of the other first recombination site.

2. A DNA molecule for the single-step cloning of two DNA segments, said molecule comprising two first site-specific recombination sites which are
(a) each functional for uptake or exchange of a DNA segment,
(b) each reactive to the same second site-specific recombination site, and,
(c) targeted by the same recombinase mix,
and wherein the recombination sequences from each of the first recombination sites can recombine with the recombination sequences of the second recombination site, without interference of the recombination sequences of the other first recombination site in view of the fact that the two first recombination sites are placed in opposite orientations in said vector.

3. A DNA molecule for the single-step cloning of two DNA segments, said molecule comprising two first site-specific recombination sites that are
(a) each comprising the same pair of recombination sequences,
(b) each functional for uptake or exchange of a DNA segment,
(c) each reactive to the same second site-specific recombination site, and,
(d) targeted by the same recombinase mix,
and wherein the recombination sequences from each of the first recombination sites can recombine with the recombination sequences of the second recombination site, without interference of the recombination sequences of the other first recombination site in view of the fact that the two first recombination sites are placed in opposite orientations in said vector.

4. A DNA molecule according to any of claims 1 to 3 wherein the first recombination sequence of said first site-specific recombination sites can only recombine with the first recombination sequence of said second recombination site and wherein the second recombination sequence of said first recombination site can only recombine with the second recombination sequence of said second recombination site in order to established directional cloning in both first recombination sites.

5. A DNA molecule according to any of claims 1 to 4 wherein said two first site-specific recombination sites comprise att recombination sequences.

6. A DNA molecule according to any of claims 1 to 5 wherein said two first site-specific recombination sites comprise attR1 and attR2 recombination sequences.

7. A DNA molecule according to any of claims 1 to 6 wherein said two first site-specific recombination sites are on the borders of a recombination cassette.

8. A DNA molecule according to any of claims 1 to 7 wherein said two first site-specific recombination sites are shortened Gateway™ cassettes, from which a part of the Chloramphenicol resistance gene has been removed.

9. A DNA molecule according to any of claims 1 to 8 wherein said second recombination site comprises att recombination sequences.

10. A DNA molecule according to any of claim 1 to 9 wherein said second recombination site comprises attL1 and attL2 recombination sequences.

11. A DNA molecule according to any of claims 1 to 10 wherein said recombination mix is a Clonase™ mix.

12. A DNA molecule according to any of claims 1 to 11 further comprising elements able to operate with the DNA segment to be cloned and which are important for the effect of said cloned DNA segments in a host cell.

13. A DNA molecule according to claim 12, wherein said elements are T-DNA borders

14. A DNA molecule according to any of claim 1 to 12 for the single step construction of a vector wherein said two first site-specific recombination sites are separated by a spacer sequence.

15. A DNA molecule according to claim 14, wherein said spacer has an insulating function.

16. A DNA molecule according to claim 14 wherein the spacer sequence comprises a MAR sequence.

17. A DNA molecule according to claim 16 wherein the spacer sequence comprises a Nicotiana tabacum MAR sequence.

18. A DNA molecule according to claim 14 wherein the spacer sequence comprises one of a GUS gene sequence, part of the sequence of the soybean promoter of the alpha′ subunit of beta-conglycinin or an intron.

19. A DNA molecule according to claim 14, wherein the spacer sequence comprises 2 separate promoters which can be identical or different, or a bidirectional promoter.

20. A DNA molecule according to claim 14, wherein the spacer sequence comprises 2 separate terminators which can be identical or different, or a bidirectional terminator.

21. A DNA molecule according to claim 14, wherein the spacer sequence is an insulating sequence and wherein the DNA molecule further comprises at least one coding sequence next to a first recombination

22. A DNA molecule according to claim 14, wherein the spacer sequence allows efficient transcription and or translation of the segments to be cloned.

23. A DNA molecule according to claim 14 wherein the spacer sequence comprises a ribosome binding site.

24. A DNA molecule according to claim 14, wherein the spacer sequence comprises a coding sequence.

25. A DNA molecule according to claim 14 wherein the spacer sequence comprises transcription initiation and/or transcription regulation elements.

26. A DNA molecule according to any of claim 1 to 25 for the high throughput cloning of two copies of the same DNA segment.

27. A DNA molecule according to any of claim 1 to 25 for the high throughput cloning of two different DNA segments

28. A DNA molecule according to any of claim 12 to 25 wherein the elements important for the effect of the cloned DNA segments are functional in prokaryotes or eukaryotes.

29. A DNA molecule according to any of claims 12 to 25 wherein the expression control elements are functional in plants.

30. A DNA molecule according to any of claim 1 to 29 wherein the first two recombination sites are comprised within T-DNA borders

31. Use of a DNA molecule according to claim 1 to 30, for the single step cloning of two different DNA segments

32. Use of a DNA molecule according to claim 1 to 30, for the single step cloning of two identical DNA segments

33. Use of a DNA molecule according to any of claim 1 to 30 for the single step construction of a co-suppression vector

34. Use of a DNA molecule according to claim 33 for the single step construction of a co-suppression vector for plants, wherein the two first recombination sites are comprised within T-DNA borders.

35. Use of a DNA molecule according to any of claims 1 to 30 for the single-step cloning of two DNA segments of interest, particularly genes or reporter genes, in each of said first site-specific recombination sites, wherein said DNA segments are identical or different, and wherein each of said two first site specific recombination sites is under the control of expression control elements, wherein said expression control elements are identical or different.

36. Use of a DNA molecule according to any of claims 1 to 30 for the single step cloning of expression control elements, particularly promoters, in each of said first site-specific recombination sites, characterized in that said DNA molecule further comprises upstream or downstream of each of said two first recombination sites, a DNA sequence of interest, particularly a gene or a reporter gene, wherein said DNA sequences are identical or different.

37. Use of a DNA molecule according to any of claims 1 to 30 for the single-step cloning of an expression control element, particularly a promoter, in one of the first site-specific recombination site and a DNA sequence of interest, particularly a gene or a reporter gene in the other first site-specific recombination site.

38. Use of a DNA molecule according to any of claim 1 to 30 for the single-step cloning of two expression control elements, particularly promoters, in opposite direction, wherein the two first recombination sites are separated by a coding region.

39. Use of a DNA molecule according to any of claim 1 to 30 for the single-step cloning of two DNA segments of interest, particularly genes, to generate a polycistronic RNA.

40. Use of a DNA molecule according to any of claim 1 to 30 for the purpose of gene stacking, by single-step cloning of two DNA segments of interest, wherein each of said DNA segments comprises at least one recombination site which is identical to at least one of said first recombination sites.

41. Use of a DNA molecule according to any of claims 1 to 30 for the single-step cloning of two DNA segments, particularly transcription control elements, to compose a bidirectional promoter.

42. Use of a DNA molecule according to any of claims 1 to 30 for the single-step cloning of two DNA segments of interest, particularly expression modules comprising a gene and a promoter.

43. Use of a DNA molecule according to any of claim 1 to 30 for the single-step cloning of two DNA segments of interest generating one or more fusion proteins.

44. A method for single-step cloning of two DNA segments at two different locations in a single vector during a single-step recombination reaction, comprising the steps of:
(a) generating a destination vector according to any of claims 1 to 30,
(b) generating one or more insert DNA segment(s) flanked with the sequences of said second site-specific recombination site, and,
(c) performing a single-step single recombination reaction by combining the destination vector and the insert segment or segments in the presence of the recombinase mix.

45. A method according to claim 44 for inserting two Identical or different copies of a DNA segment for obtaining one of a co-suppression vector, a gene combination vector, a promoter combination vector, a promoter-gene combination vector, a gene silencing vector, a polycistronic vector, a gene stacking vector, or a vector comprising a bidirectional promoter or combinatorial expression cassettes, or a fusion protein.

46. A DNA molecule obtainable by a method according to claim 44 or 45.

47. A transgenic host cell comprising a DNA molecule according to any of claim 1 to 30.

48. A transgenic plant, in particular a crop plant, comprising a DNA molecule according to any of claim 1 to 30.

49. A transgenic non-human mammalian organism comprising a DNA molecule according to any of claim 1 to 30.

50. A transgenic host organism selected from the group comprising prokaryotes, eukaryotes, animal, fish, insects, yeast, mould, fungi, nematodes, comprising a DNA molecule according to any of claim 1 to 30.
